(19) United States
(12) Patent Application Publication
(10) Pub. No.: US 2019/0224841 A1
(43) Pub. Date: Jul. 25, 2019

(54) EXOSUIT SYSTEMS AND METHODS FOR MONITORING WORKING SAFETY AND PERFORMANCE

(71) Applicant: SEISMIC HOLDINGS, INC., Menlo Park, CA (US)

(72) Inventors: Daniel Le Ly, Mountain View, CA (US); Ray Franklin Cowan, Mountain View, CA (US); Andrew Robert Chang, Sunnyvale, CA (US); Richard Mahoney, Los Altos, CA (US)

(21) Appl. No.: 16/256,812

(22) Filed: Jan. 24, 2019

Related U.S. Application Data
(60) Provisional application No. 62/621,464, filed on Jan. 24, 2018.
(60) Provisional application No. 62/668,262, filed on May 8, 2018.
(60) Provisional application No. 62/682,179, filed on Jun. 8, 2018.

(51) Int. Cl. B25J 9/00; A61B 5/11; A61B 5/00

(52) U.S. Cl. CPC B25J 9/0006; A61B 5/6835; A61B 5/6813; A61B 5/1126

(57) ABSTRACT

Exosuit systems and methods according to various embodiments are described herein. The exosuit can be used to monitor users for injury, perform injury prevention, and monitor works for productivity.

### CROSS-REFERENCE TO RELATED APPLICATIONS

This application claims priority to U.S. Provisional Patent Application Nos. 62/621,464, filed Jan. 24, 2018, 62/668,262, filed May 8, 2018, and 62/682,179, filed Jun. 8, 2018, which are incorporated by reference in their entireties.

### TECHNICAL FIELD

This disclosure relates generally to the field of worker monitoring technology, and more specifically to systems and methods for monitoring worker safety and productivity.

### BACKGROUND

Wearable robotic systems have been developed for augmentation of humans' natural capabilities, or to replace functionality lost due to injury or illness. Employees engaged in physical labor may use wearable robotic systems to perform their jobs. When workers are engaged in work, it may be desirable to monitor them for safety and performance.

### SUMMARY

Systems and methods for monitoring worker safety and productivity are discussed herein. Exosuits worn by users can monitor several movement factors that characterize the user's movement and any changes in the users movement with a high degree of specificity that enables various control system algorithms to assess whether the exosuit user is performing work-related moves correctly (e.g., such as heavy lifting), is fatigued, or is injured, and the exosuit can provide the appropriate feedback in response to the basement. In addition, the exosuits can be used to assist in the enforcement of workplace policies and laws.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIGS. 1A-1C show front, back, and side views of a base layer of an exosuit according to an embodiment;

FIGS. 1D-1F show front, back, and side views, respectively, of a power layer according to an embodiment;

FIGS. 1G and 1H show respective front and back views of a human male's musculature anatomy, according to an embodiment;

FIGS. 1I and 1J show front and side views of an illustrative exosuit having several power layer segments that approximate many of the muscles shown in FIGS. 1G and 1H, according to various embodiments;

FIGS. 2A and 2B show front and back view of illustrative exosuit according to an embodiment;

FIG. 3 shows an illustrative symbiosis exosuit system according to an embodiment;

FIG. 4 shows illustrative process for implementing a symbiosis exosuit system according to an embodiment;

FIG. 5 shows an illustrative diagram of different control modules that may be implemented by an exosuit according to an embodiment;

FIG. 6 shows an illustrative block diagram according to an embodiment;

FIG. 7 shows illustrative training process according to an embodiment;

FIGS. 8A-8D show illustrative injury detection process according to an embodiment;

FIGS. 9A-9C show illustrative worker productivity process according to an embodiment;

FIG. 10 shows illustrative equipment operating process according to an embodiment;

FIG. 11 shows illustrative worker movement monitoring process according to an embodiment;

FIG. 12 shows illustrative maximizing worker productivity process according to an embodiment;

FIG. 13 shows illustrative policy enforcement process according to an embodiment;

FIG. 14 shows illustrative job fitness determination process according to an embodiment;

FIG. 15A shows illustrative vibration monitoring process according to an embodiment;

FIG. 15B shows an illustrative force profile;

FIG. 15C shows an illustrative table;

FIG. 16A shows illustrative process for using an exosuit to assist a user according to an embodiment;

FIG. 16B shows movement factor data corresponding to a person before injury (or fatigue) and after injury (or fatigue);

FIGS. 17A and 17B show illustrative “before” and “after” plots of movement data for an assembly-line worker according to an embodiment;

FIG. 18 illustrates an example exosuit according to an embodiment; and

FIG. 19 is a schematic illustrating elements of an exosuit and a hierarchy of control or operating the exosuit according to an embodiment;

### DETAILED DESCRIPTION

In the following description, numerous specific details are set forth regarding the systems, methods and media of the disclosed subject matter and the environment in which such systems, methods and media may operate, etc., in order to provide a thorough understanding of the disclosed subject matter. It can be apparent to one skilled in the art, however, that the disclosed subject matter may be practiced without such specific details, and that certain features, which are well known in the art, are not described in detail in order to avoid complication of the disclosed subject matter. In addition, it can be understood that the examples provided below are exemplary, and that it is contemplated that there are other systems, methods and media that are within the scope of the disclosed subject matter.

In the descriptions that follow, an exosuit or assistive exosuit is a suit that is worn by a wearer on the outside of his or her body. It may be worn under the wearer's normal clothing, over their clothing, between layers of clothing, or may be the wearer's primary clothing itself. The exosuit may be supportive and/or assistive, as it physically supports or assists the wearer in performing particular activities, or can provide other functionality such as communication to the wearer through physical expressions to the body, engagement of the environment, or capturing of information from the wearer. In some embodiments, a powered exosuit system can include several subsystems, or layers. In some embodiments, the powered exosuit system can include more or less subsystems or layers. The subsystems or layers can include the base layer, stability layer, power layer, sensor and controls layer, a covering layer, and user interface/user experience (UI/UX) layer.

The base layer provides the interfaces between the exosuit system and the wearer's body. The base layer may be adapted to be worn directly against the wearer's skin, between undergarments and outer layers of clothing, over outer layers of clothing or a combination thereof, or the base layer may be designed to be worn as primary clothing itself. In some embodiments, the base layer can be adapted to be both comfortable and unobtrusive, as well as to comfortably and efficiently transmit loads from the stability layer and power layer to the wearer's body in order to provide the desired assistance. The base layer can typically comprise several different material types to achieve these purposes. Elastic materials may provide compliance to conform to the wearer's body and allow for ranges of movement. The innermost layer is typically adapted to grip the wearer's skin, undergarments or clothing so that the base layer does not slip as loads are applied. Substantially inextensible materials may be used to transfer loads from the stability layer and power layer to the wearer's body. These materials may be substantially inextensible in one axis, yet flexible or extensible in other axes such that the load transmission is along preferred paths. The load transmission paths may be optimized to distribute the loads across regions of the wearer's body to minimize the forces felt by the wearer, while providing efficient load transfer with minimal loss and not causing the base layer to slip. Collectively, this load transmission configuration within the base layer may be referred to as a load distribution member. Load distribution members refer to flexible elements that distribute loads across a region of the wearer's body. Examples of load distribution members can be found in International Application PCT/US16/19565, titled “Flexgrip,” (published as WO 2016/138264) the contents of which are incorporated herein by reference.

The load distribution members may incorporate one or more catenary curves to distribute loads across the wearer's body. Multiple load distribution members or catenary curves may be joined with pivot points, such that as loads are applied to the structure, the arrangement of the load distribution members pivots tightens or constricts on the body to increase the gripping strength. Compressive elements such as battens, rods, or stays may be used to transfer loads to different areas of the base layer for comfort or structural purposes. For example, a power layer component may terminate in the middle back due to its size and orientation requirements, however the load distribution members that anchor the power layer component may reside on the lower back. In this case, one or more compressive elements may transfer the load from the power layer component at the middle back to the load distribution member at the lower back.

The load distribution members may be constructed using multiple fabrication and textile application techniques. For example, the load distribution member can be constructed from a layered woven 45°/90° with bonded edge, spandex tooth, organza (poly) woven 45°/90° with bonded edge, organza (cotton/silk) woven 45°/90°, and Tyvek (non-woven). The load distribution member may be constructed using knit and lacing or horse hair and spandex tooth. The load distribution member may be constructed using channels and/or laces.

The base layer may include a flexible underlayer that is constructed to compress against a portion of the wearer's body, either directly to the skin, or to a clothing layer, and also provides a relatively high grip surface for one or more load distribution members to attach thereto. The load distribution members can be coupled to the underlayer to facilitate transmission of shears or other forces from the members, via the flexible underlayer, to skin of a body segment or to clothing worn over the body segment, to maintain the trajectories of the members relative to such a body segment, or to provide some other functionality. Such a flexible underlayer could have a flexibility and/or compliance that differs from that of the member (e.g., that is less than that of the members, at least in a direction along the members), such that the member can transmit forces along their length and evenly distribute shear forces and/or pressures, via the flexible underlayer, to skin of a body segment to which a flexible body harness is mounted.

Further, such a flexible underlayer can be configured to provide additional functionality. The material of the flexible underlayer could include anti-bacterial, anti-fungal, or other agents (e.g., silver nanoparticles) to prevent the growth of microorganisms. The flexible underlayer can be configured to manage the transport of heat and/or moisture (e.g., sweat) from a wearer to improve the comfort and efficiency of activity of the wearer. The flexible underlayer can include straps, seams, hook-and-loop fasteners, clasps, zippers, or other elements configured to maintain a specified relationship between elements of the load distribution members and aspects of a wearer's anatomy. The underlayer can additionally increase the ease with which a wearer can don and/or doff the flexible body harness and/or a system (e.g., a flexible exosuit system) or garment that includes the flexible body harness. The underlayer can additionally be configured to protect the wearer from ballistic weapons, sharp edges, shrapnel, or other environmental hazards (by including, e.g., panels or flexible elements of para-aramid or other high-strength materials).

The base layer can additionally include features such as size adjustments, openings and electro-mechanical integration features to improve ease of use and comfort for the wearer.

Size adjustment features permit the exosuit to be adjusted to the wearer's body. The size adjustments may allow the suit to be tightened or loosened about the length or circumference of the torso or limbs. The adjustments may comprise lacing, the Boa system, webbing, elastic, hook-and-loop or other fasteners. Size adjustment may be accomplished by the load distribution members themselves, as they constrict onto the wearer when loaded. In one example, the torso circumference may be tightened with corset-style lacing, the legs tightened with hook-and-loop in a double-back configuration, and the length and shoulder height adjusted with webbing and tension-lock fasteners such as cam-locks, D-rings or the like. The size adjustment features in the base layer may be actuated by the power layer to dynamically adjust the base layer to the wearer's body in different positions, in order to maintain consistent pressure and comfort for the wearer. For example, the base layer may be required to tighten on the thighs when standing, and loosen when sitting such that the base layer does not excessively constrict the thighs when seated. The dynamic size adjustment may be controlled by the sensor and controls layer, for example by detecting pressures or forces in the base layer and actuating the power layer to consistently attain the desired force or pressure. This feature does not necessarily cause the suit to provide physical assistance, but can create a more comfortable experience for the wearer, or allow the physical assistance elements of the suit to perform better or differently depending on the purpose of the movement assistance.

Opening features in the base layer may be provided to facilitate donning (putting the exosuit on) and doffing (taking the exosuit off) for the wearer. Opening features may comprise zippers, hook-and-loop, snaps, buttons or other textile fasteners. In one example, a front, central zipper provides an opening feature for the torso, while hook-and-loop fasteners provide opening features for the legs and shoulders. In this case, the hook-and-loop fasteners provide both opening and adjustment features. In other examples, the exosuit may simply have large openings, for example around the arms or neck, and elastic panels that allow the suit to be donned and doffed without specific closure mechanisms. A truncated load distribution member may be simply extended to tighten on the wearer's body. Openings may be provided to facilitate toileting so the user can keep the exosuit on, but only have to remove or open a relatively small portion to use the bathroom.

Electro-mechanical integration features attach components of the stability layer, power layer and sensor and controls layer into the base layer for integration into the exosuit. The integration features may be for mechanical, structural, comfort, protective or cosmetic purposes. Structural integration features anchor components of the other layers to the base layer. For the stability and power layers, the structural integration features provide for load-transmission to the base layer and load distribution members, and may accommodate specific degrees of freedom at the attachment point. For example, a snap or rivet anchoring a stability or power layer element may provide both load transmission to the base layer, as well as a pivoting degree of freedom. Stitched, adhesive, or bonded anchors may provide load transmission with or without the pivoting degree of freedom. A sliding anchor, for example along a sleeve or rail, may provide a translational degree of freedom. Anchors may be separable, such as with snaps, buckles, clasps or hooks; or may be inseparable, such as with stitching, adhesives or other bonding. Size adjustment features as described above may allow adjustment and customization of the stability and power layers, for example to adjust the tension of spring or elastic elements in the passive layer, or to adjust the length of actuators in the power layer.

Other integration features such as loops, pockets, and mounting hardware may simply provide attachment to components that do not have significant load transmission requirements, such as batteries, circuit boards, sensors, or cables. In some cases, components may be directly integrated into textile components of the base layer. For example, cables or connectors may include conductive elements that are directly woven, bonded or otherwise integrated into the base layer.

Electromechanical integration features may also protect or cosmetically hide components of the stability, power or sensor and controls layers. Elements of the stability layer (e.g. elastic bands or springs), power layer (e.g. flexible linear actuators or twisted string actuators) or sensor and controls layer (e.g. cables) may travel through sleeves, tubes, or channels integrated into the base layer, which can both conceal and protect these components. The sleeves, tubes, or channels may also permit motion of the component, for example during actuation of a power layer element. The sleeves, channels, or tubes may comprise resistance to collapse, ensuring that the component remains free and uninhibited within.

Enclosures, padding, fabric coverings, or the like may be used to further integrate components of other layers into the base layer for cosmetic, comfort, or protective purposes. For example, components such as motors, batteries, cables, or circuit boards may be housed within an enclosure, fully or partially covered or surrounded in padded material such that the components do not cause discomfort to the wearer, are visually unobtrusive and integrated into the exosuit, and are protected from the environment. Opening and closing features may additionally provide access to these components for service, removal, or replacement.

In some cases—particularly for exosuits configurable for either provisional use or testing—a tether may allow for some electronic and mechanical components to be housed off the suit. In one example, electronics such as circuit boards and batteries may be over-sized, to allow for added configurability or data capture. If the large size of these components makes it undesirable to mount them on the exosuit, they could be located separately from the suit and connected via a physical or wireless tether. Larger, over-powered motors may be attached to the suit via flexible drive linkages that allow actuation of the power layer without requiring large motors to be attached to the suit. Such over-powered configurations allow optimization of exosuit parameters without constraints requiring all components to be attached or integrated into the exosuit.

Electro-mechanical integration features may also include wireless communication. For example, one or more power layer components may be placed at different locations on the exosuit. Rather than utilizing physical electrical connections to the sensors and controls layer, the sensor and controls layer may communicate with the one or more power layer components via wireless communication protocols such as Bluetooth, ZigBee, ultrawide band, or any other suitable communication protocol. This may reduce the electrical interconnections required within the suit. Each of the one or more power layer components may additionally incorporate a local battery such that each power layer component or group of power layer components are independently powered units that do not require direct electrical interconnections to other areas of the exosuit.

The stability layer provides passive mechanical stability and assistance to the wearer. The stability layer comprises one or more passive (non-powered) spring or elastic elements that generate forces or store energy to provide stability or assistance to the wearer. An elastic element can have an un-deformed, least-energy state. Deformation, e.g. elongation, of the elastic element stores energy and generates a force oriented to return the elastic element toward its least-energy state. For example, elastic elements approximating hip flexors and hip extensors may provide stability to the wearer in a standing position. As the wearer deviates from the standing position, the elastic elements are deformed, generating forces that stabilize the wearer and assist maintaining the standing position. In another example, as a wearer moves from a standing to seated posture, energy is stored in one or more elastic elements, generating a restorative force to assist the wearer when moving from the seated to standing position. Similar passive, elastic elements may be adapted to the torso or other areas of the limbs to provide positional stability or assistance moving to a position where the elastic elements are in their least-energy state.

Elastic elements of the stability layer may be integrated to parts of the base layer or be an integral part of the base layer. For example elastic fabrics containing spandex or similar materials may serve as a combination base/stability layer. Elastic elements may also include discrete components such as springs or segments of elastic material such as silicone or elastic webbing, anchored to the base layer for load transmission at discrete points, as described above.

The stability layer may be adjusted as described above, both to adapt to the wearer's size and individual anatomy, as well as to achieve a desired amount of pre-tension or slack in components of the stability layer in specific positions. For example, some wearers may prefer more pre-tension to provide additional stability in the standing posture, while others may prefer more slack, so that the passive layer does not interfere with other activities such as ambulation.

The stability layer may interface with the power layer to engage, disengage, or adjust the tension or slack in one or more elastic elements. In one example, when the wearer is in a standing position, the power layer may pre-tension one or more elastic elements of the stability layer to a desired amount for maintaining stability in that position. The pre-tension may be further adjusted by the power layer for different positions or activities. In some embodiments, the elastic elements of the stability layer should be able to generate at least 5 lbs force; preferably at least 50 lbs force when elongated.

The power layer can provide active, powered assistance to the wearer, as well as electromechanical clutching to maintain components of the power or stability layers in a desired position or tension. The power layer can include one or more flexible linear actuators (FLA). An FLA is a powered actuator capable of generating a tensile force between two attachment points, over a give stroke length. An FLA is flexible, such that it can follow a contour, for example around a body surface, and therefore the forces at the attachment points are not necessarily aligned. In some embodiments, one or more FLAs can include one or more twisted string actuators. In the descriptions that follow, FLA refers to a flexible linear actuator that exerts a tensile force, contracts or shortens when actuated. The FLA may be used in conjunction with a mechanical clutch that locks the tension force generated by the FLA in place so that the FLA motor does not have to consume power to maintain the desired tension force. Examples of such mechanical clutches are discussed below. In some embodiments, FLAs can include one or more twisted string actuators or flexdrives, as described in further detail in U.S. Pat. No. 9,266,233, titled “Exosuit System,” the contents of which are incorporated herein by reference. FLAs may also be used in connection with electrolaminate clutches, which are also described in the U.S. Pat. No. 9,266,233. The electrolaminate clutch (e.g., clutches configured to use electrostatic attraction to generate controllable forces between clutching elements) may provide power savings by locking a tension force without requiring the FLA to maintain the same force.

The powered actuators, or FLAs, are arranged on the base layer, connecting different points on the body, to generate forces for assistance with various activities. The arrangement can often approximate the wearer's muscles, in order to naturally mimic and assist the wearer's own capabilities. For example, one or more FLAs may connect the back of the torso to the back of the legs, thus approximating the wearer's hip extensor muscles. Actuators approximating the hip extensors may assist with activities such as standing from a seated position, sitting from a standing position, walking, or lifting. Similarly, one or more actuators may be arranged approximating other muscle groups, such as the hip flexors, spinal extensors, abdominal muscles or muscles of the arms or legs.

The one or more FLAs approximating a group of muscles are capable of generating at least 10 lbs over at least a ½ inch stroke length within 4 seconds. In some embodiments, one or more FLAs approximating a group of muscles may be capable of generating at least 250 lbs over a 6-inch stroke within ½ second. Multiple FLAs, arranged in series or parallel, may be used to approximate a single group of muscles, with the size, length, power, and strength of the FLAs optimized for the group of muscles and activities for which they are utilized.

The sensor and controls layer captures data from the suit and wearer, utilizes the sensor data and other commands to control the power layer based on the activity being performed, and provides suit and wearer data to the UX/UI layer for control and informational purposes.

Sensors such as encoders or potentiometers may measure the length and rotation of the FLAs, while force sensors measure the forces applied by the FLAs. Inertial measurement units (IMUs) measure and enable computation of kinematic data (positions, velocities and accelerations) of points on the suit and wearer. These data enable inverse dynamics calculations of kinetic information (forces, torques) of the suit and wearer. Electromyographic (EMG) sensors may detect the wearer's muscle activity in specific muscle groups. Electronic control systems (ECSs) on the suit may use parameters measured by the sensor layer to control the power layer. Data from the IMUs may indicate both the activity being performed, as well as the speed and intensity. For example, a pattern of IMU or EMG data may enable the ECS to detect that the wearer is walking at a specific pace. This information then enables the ECS, utilizing the sensor data, to control the power layer in order to provide the appropriate assistance to the wearer. Stretchable sensors may be used as a strain gauge to measure the strain of the elements in the stability layer, and thereby predict the forces in the elastic elements of the stability layer. Stretchable sensors may be embedded in the base layer or grip layer and used to measure the motion of the fabrics in the base layer and the motion of the body.

Data from the sensor layer may be further provided to the UX/UI layer, for feedback and information to the wearer, caregivers or service providers.

The UX/UI layer comprises the wearer's and others' interaction and experience with the exosuit system. This layer includes controls of the suit itself such as initiation of activities, as well as feedback to the wearer and caregivers. A retail or service experience may include steps of fitting, calibration, training and maintenance of the exosuit system. Other UX/UI features may include additional lifestyle features such as electronic security, identity protection and health status monitoring.

The assistive exosuit can have a user interface for the wearer to instruct the suit which activity is to be performed, as well as the timing of the activity. In one example, a user may manually instruct the exosuit to enter an activity mode via one or more buttons, a keypad, or a tethered device such as a mobile phone. In another example, the exosuit may detect initiation of an activity from the sensor and controls layer, as described previously. In yet another example, the user may speak a desired activity mode to the suit, which can interpret the spoken request to set the desired mode. The suit may be pre-programmed to perform the activity for a specific duration, until another command is received from the wearer, or until the suit detects that the wearer has ceased the activity. The suit may include cease activity features that, when activated, cause the suit to cease all activity. The cease activity features can take into account the motion being performed, and can disengage in a way that takes into account the user's position and motion, and safely returns the user to an unloaded state in a safe posture.

The exosuit may have a UX/UI controller that is defined as a node on another user device, such as a computer or mobile smart phone. The exosuit may also be the base for other accessories. For example, the exosuit may include a cell phone chip so that the suit may be capable of receiving both data and voice commands directly similar to a cell phone, and can communicate information and voice signals through such a node. The exosuit control architecture can be configured to allow for other devices to be added as accessories to the exosuit. For example, a video screen may be connected to the exosuit to show images that are related to the use of the suit. The exosuit may be used to interact with smart household devices such as door locks or can be used to turn on smart televisions and adjust channels and other settings. In these modes, the physical assist of the suit can be used to augment or create physical or haptic experiences for the wearer that are related to communication with these devices. For instance, an email could have a pat on the back as a form of physical emoji that when inserted in the email causes the suit to physically tap the wearer or perform some other type of physical expression to the user that adds emphasis to the written email.

The exosuit may provide visual, audio, or haptic feedback or cues to inform the user of various exosuit operations. For example, the exosuit may include vibration motors to provide haptic feedback. As a specific example, two haptic motors may be positioned near the front hip bones to inform the user of suit activity when performing a sit-to-stand assistive movement. In addition, two haptic motors may be positioned near the back hip bones to inform the user of suit activity when performing a stand-to-sit assistive movement. The exosuit may include one or more light emitting diodes (LEDs) to provide visual feedback or cues. For example, LEDS may be placed near the left and/or right shoulders within the peripheral vision of the user. The exosuit may include a speaker or buzzer to provide audio feedback or cues.

In other instances, the interaction of the FLA's with the body through the body harness and otherwise can be used as a form of haptic feedback to the wearer, where changes in the timing of the contraction of the FLA's can indicate certain information to the wearer. For instance, the number or strength of tugs of the FLA on the waist could indicate the amount of battery life remaining or that the suit has entered a ready state for an impending motion.

The control of the exosuit may also be linked to the sensors that are measuring the movement of the wearer, or other sensors, for instance on the suit of another person, or sensors in the environment. The motor commands described herein may all be activated or modified by this sensor information. In this example, the suit can exhibit its own reflexes such that the wearer, through intentional or unintentional motions, cues the motion profile of the suit. When sitting, for further example, the physical movement of leaning forward in the chair, as if to indicate an intention to stand up, can be sensed by the suit IMU's and be used to trigger the sit to stand motion profile. In one embodiment, the exosuit may include sensors (e.g., electroencephalograph (EEG) sensor) that are able to monitor brain activity may be used to detect a user's desire to perform a particular movement. For example, if the user is sitting down, the EEG sensor may sense the user's desire to stand up and cause the exosuit to prime itself to assist the user in a sit-to-stand assistive movement.

The suit may make sounds or provide other feedback, for instance through quick movements of the motors, as information to the user that the suit has received a command or to describe to the user that a particular motion profile can be applied. In the above reflex control example, the suit may provide a high pitch sound and/or a vibration to the wearer to indicate that it is about to start the movement. This information can help the user to be ready for the suit movements, improving performance and safety. Many types of cues are possible for all movements of the suit.

Control of the suit includes the use of machine learning techniques to measure movement performance across many instances of one or of many wearers of suits connected via the internet, where the calculation of the best control motion for optimizing performance and improving safety for any one user is based on the aggregate information in all or a subset of the wearers of the suit. The machine learning techniques can be used to provide user specific customization for exosuit assistive movements. For example, a particular user may have an abnormal gait (e.g., due to a car accident) and thus is unable to take even strides. The machine learning may detect this abnormal gait and compensate accordingly for it.

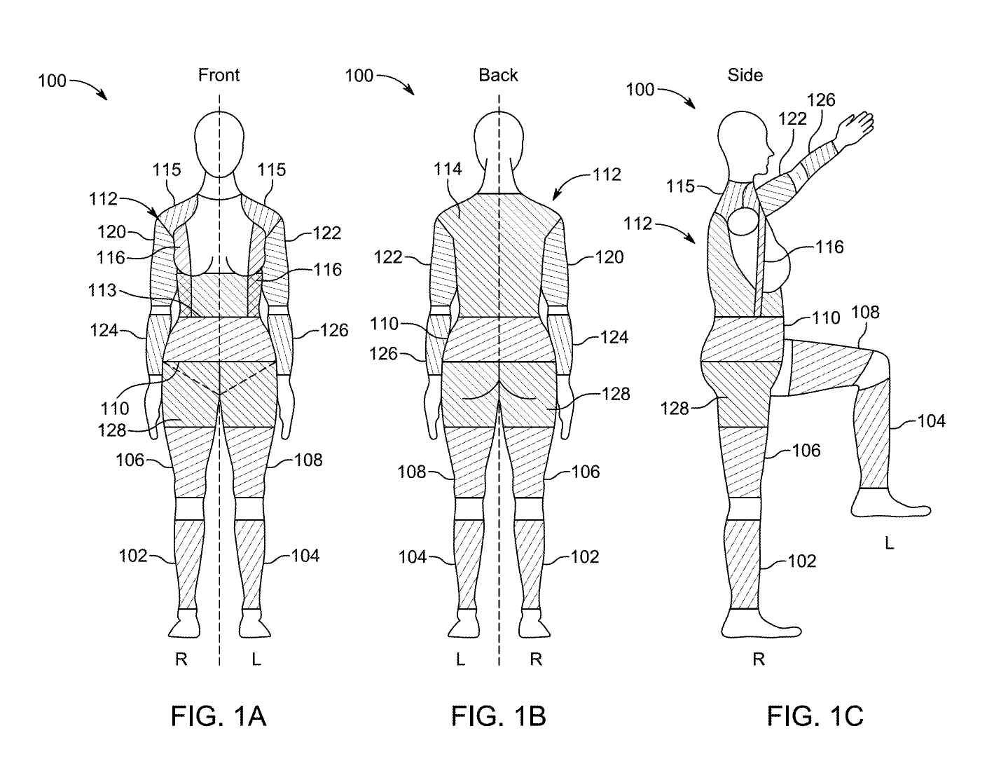

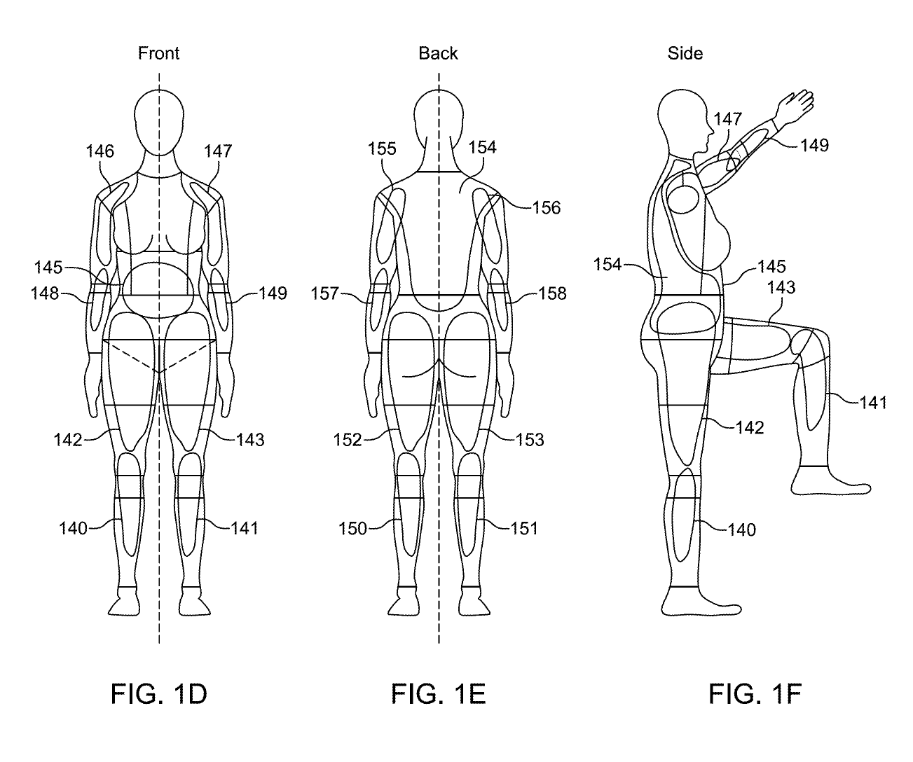

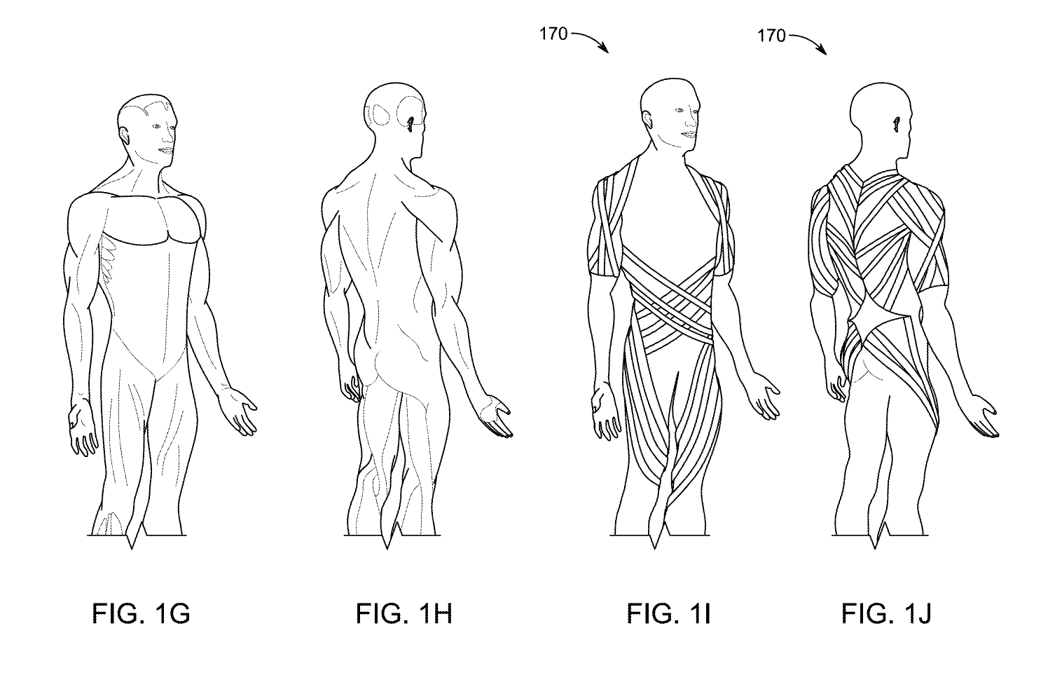

FIGS. 1A-1C show front, back, and side views of a base layer 100 of an exosuit according to an embodiment. Base layer 100 may be worn as a single piece or as multiple pieces. As shown, base layer 100 is shown to represent multiple pieces that can serve as load distribution members (LDMs) for the power layer (shown in FIGS. 1D-1F). Base layer 100 and any LDMs thereof can cover or occupy any part of the human body as desired. The LDMs shown in FIGS. 1A-1C are merely illustrative of a few potential locations and it should be appreciated that additional LDMs may be added or certain LDMs may be omitted.

Base layer 100 can include calf LDMs 102 and 104 that are secured around the calf region or lower leg portion of the human. Calf LDMs 102 and 104 are shown to be positioned between the knees and the ankles, but this is merely illustrative. If desired, calf LDM 102 and 104 can also cover the foot and ankle and/or the knee.

Base layer 100 can include thigh LDMs 106 and 108 that are secured around the thigh region of the human. Thigh LDMs 106 and 108 are shown to be positioned between the knees and an upper region of the thighs. In some embodiments, thigh LDMs 106 and 108 and calf LDMs 102 and 104, respectively, may be merged together to form leg LDMs that cover the entirety of the legs and/or feet.

Base layer 100 can include hip LDM 110 that is secured around a hip region of the human. LDM 110 may be bounded such that it remains positioned above the toileting regions of the human. Such bounding may make toileting relatively easy for the human as he or she would be not be required to remove base layer 100 to use the bathroom. In some embodiments, LDM 110 may be attached to thigh LDMs 106 and 108, but the toileting regions may remain uncovered. In another embodiment, a removable base layer portion may exist between LDM 100 and thigh LDMS 106 and 108.

Base layer 100 can include upper torso LDM 112 that is secured around an upper torso region of the human. Upper torso LDM 112 may include waist LDM 113, back LDM 114, shoulder LDM 115, and shoulder strap LDMs 116. Waist LDM 113, back LDM 114, shoulder LDM 115, and shoulder strap LDMs 116 may be integrally formed to yield upper torso LDM 112. In some embodiments, a chest LDM (not shown) may also be integrated into upper torso LDM 112. Female specific exosuits may have built in bust support for the chest LDM.

Base layer 100 can include upper arm LDMs 120 and 122 and lower arm LDMs 124 and 126. Upper arm LDMs 120 and 122 may be secured around bicep/triceps region of the arm and can occupy space between the shoulder and the elbow. Lower arm LDMs 124 and 126 may be secured around the forearm region of the arm and can occupy the space between the elbow and the wrist. If desired, upper arm LDM 120 and lower arm LDM 124 may be integrated to form an arm LDM, and upper arm LDM 122 and lower arm LDM 126 may be integrated to form another arm LDM. In some embodiments, arm LDMS 120, 122, 124, and 126 may form part of upper torso LDM 112.

Base layer 100 can include gluteal/pelvic LDM 128 that is secured the gluteal and pelvic region of the human. LDM 128 may be positioned between thigh LDMs 106 and 108 and hip LDM 110. LDM 128 may have removable portions such as buttoned or zippered flaps that permit toileting. Although not shown in FIGS. 1A-1C, LDMs may exist for the feet, toes, neck, head, hands, fingers, elbows, or any other suitable body part.

As explained above, the LDMs may serve as attachment points for components of the power layer. In particular, the components that provide muscle assistance movements typically need to be secured in at least two locations on the body. This way, when the flexible linear actuators are engaged, the contraction of the actuator can apply a force between the at least two locations on the body. With LDMs strategically placed around the body, the power layer can also be strategically placed thereon to provide any number of muscle assistance movements. For example, the power layer may be distributed across different LDMs or within different regions of the same LDM to approximate any number of different muscles or muscle groups. The power layer may approximate muscle groups such as the abdominals, adductors, dorsal muscles, shoulders, arm extensors, wrist extensors, gluteals, arm flexors, wrist flexors, scapulae fixers, thigh flexors, lumbar muscles, surae, pectorals, quadriceps, and trapezii.

FIGS. 1D-1F show front, back, and side views, respectively, of a power layer according to an embodiment. The power layer is shown as multiple segments distributed across and within the various LDMs. As shown, the power layer can include power layer segments 140-158. Each of power layer segments can include any number of flexible linear actuators. Some of the power layer segments may exist solely on the anterior side of the body, exist solely on the posterior side, start on the anterior side and wrap around to the posterior side, start on the posterior side and wrap around to the anterior side, or wrap completely around a portion of the body. Power layer segment (PLS) 140 may be secured to LDM 102 and LDM 106, and PLS 141 may be secured to LDM 104 and LDM 108. PLS 142 may be secured to LDM 106 and LDM 110 and/or LDM 114, and PLS 143 may be secured to LDM 108 and LDM 110 and/or LDM 114. PLS 145 may be secured to LDM 110 and LDM 113 and/or to LDM 114 or LDM 128. PLS 146 may be secured to LDM 115 and LDM 120, and PLS 147 may be secured to LDM 115 and LDM 122. PLS 148 may be secured to LDM 120 and LDM 124, and PLS 149 may be secured to LDM 122 and LDM 126.

PLS 150 may be secured to LDM 104 and LDM 108, and PLS 151 may be secured to LDM 102 and LDM 106. PLS 152 may be secured to LDM 106 and LDM 110 and/or to LDM 113, and PLS 153 may be secured to LDM 108 and LDM 110 and/or LDM 113. PLS 154 may be secured to LDM 112 and LDM 110. PLS 155 may be secured to LDM 112 and LDM 120, and PLS 156 may be secured to LDM 112 and LDM 122. PLS 157 may be secured to LDM 120 and LDM 124, and PLS 158 may be secured to LDM 122 and LDM 126.

It should be appreciated that the power layer segments are merely illustrative and that additional power layer segments may be added or that some segments may be omitted. In addition, the attachment points for the power layer segments are merely illustrative and that other attachment points may be used.

The human body has many muscles, including large and small muscles that are arranged in all sorts of different configuration. For example, FIGS. 1G and 1H show respective front and back views of a human male's musculature anatomy, which shows many muscles. In particular, the abdominals, adductors, dorsal muscles, shoulders, arm extensors, wrist extensors, gluteals, arm flexors, wrist flexors, scapulae fixers, thigh flexors, lumbar muscles, pectorals, quadriceps, and trapezii are all shown.

The LDMs may be designed so that they can accommodate different sizes of individuals who don the exosuit. For example, the LDMs may be adjusted to achieve the best fit. In addition the LDMs are designed such that the location of the end points and the lines of action are co-located with the bone structure of the user in such a way that the flexdrive placement on the exosuit system are aligned with the actual muscle structure of the wearer for comfort, and the moment arms and forces generated by the flexdrive/exosuit system feel aligned with the forces generated by the wearer's own muscles.

FIGS. 1I and 1J show front and side views of illustrative exosuit 170 having several power layer segments that approximate many of the muscles shown in FIGS. 1G and 1H. The power layer segments are represented by the individual lines that span different parts of the body. These lines may represent specific flexible linear actuators or groups thereof that work together to form the power layer segments that are secured to the LDMs (not shown). As shown, the FLAs may be arrayed to replicate at least a portion of each of the abdominal muscles, dorsal muscles, shoulder muscles, arm extensor and flexor muscles, gluteal muscles, quadriceps muscles, thigh flexor muscles, and trapezii muscles. Thus, exosuit 170 exemplifies one of many possible different power layer segment arrangements that may be used in exosuits in accordance with embodiments discussed herein. These power layer segments are arranged so that the moment arms and forces generated feel like forces being generated by the user's own muscles, tendons, and skeletal structure. Other possible power layer segment arrangements are illustrated and discussed below.

The power layer segments may be arranged such that they include opposing pairs or groups, similar to the way human muscles are arranged in opposing pairs or groups of muscles. That is, for a particular movement, the opposing pairs or groups can include protagonist and antagonist muscles. While performing the movement, protagonist muscles may perform the work, whereas the antagonist muscles provide stabilization and resistance to the movement. As a specific example, when a user is performing a curl, the biceps muscles may serve as the protagonist muscles and the triceps muscles may serve as the antagonist muscles. In this example, the power layer segments of an exosuit may emulate the biceps and triceps. When the biceps human muscle is pulling to bend the elbow, the exosuit triceps power layer segment can pull on the other side of the joint to resist bending of the elbow by attempting to extend it. The power layer segment can be, for example, either be a FLA operating alone to apply the force and motion, or a FLA in series with an elastic element. In the latter case, the human biceps would be working against the elastic element, with the FLA adjusting the length and thereby the resistive force of the elastic element.

Thus, by arranging the power layer segments in protagonist and antagonist pairs, the power layers segments can mimic or emulate any protagonist and antagonist pairs of the human anatomy musculature system. This can be used to enable exosuits to provide assistive movements, alignment movements, and resistive movements. For example, for any exercise movement requires activation of protagonist muscles, a subset of the power layer segments can emulate activation of antagonist muscles associated with that exercise movement to provide resistance.

The design flexibility of the LDMs and PLSs can enable exosuits to be constructed in accordance with embodiments discussed herein. Using exosuits, the power layer segments can be used to resist motion, assist motion, or align the user's form.

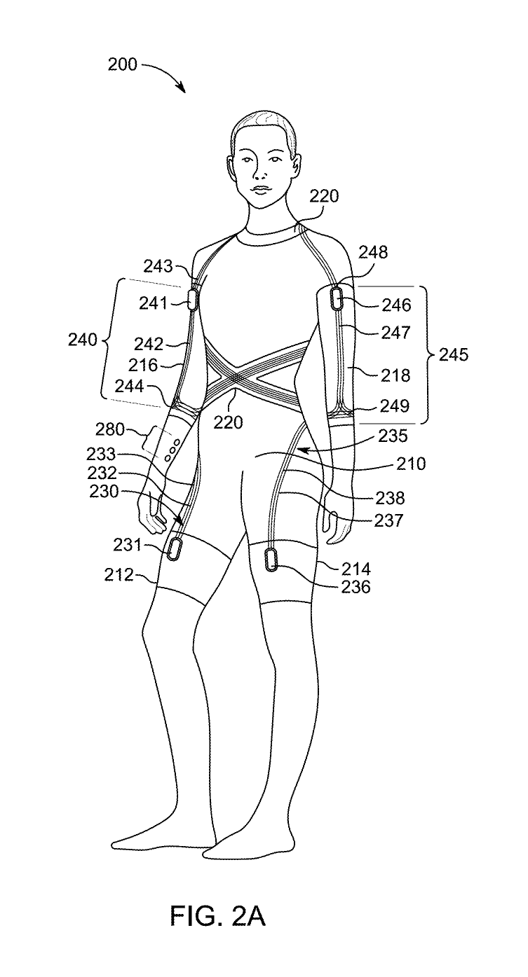

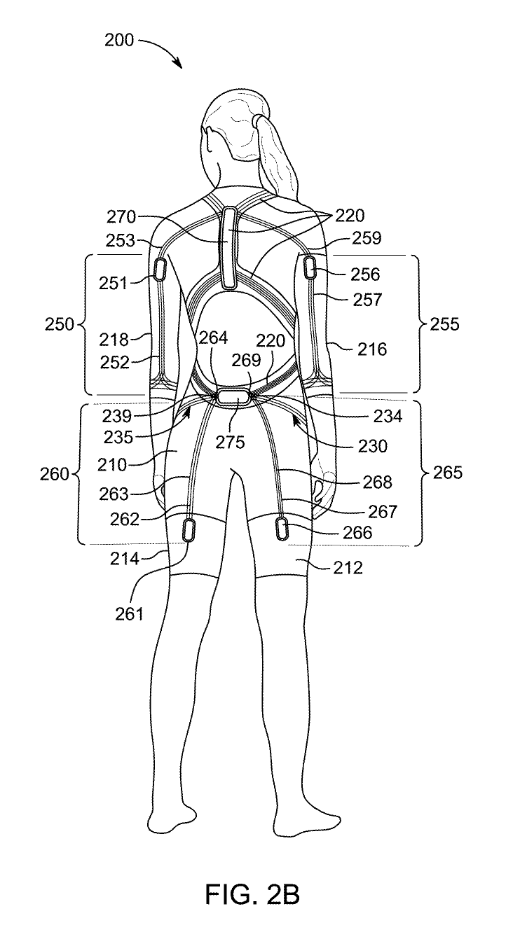

FIGS. 2A and 2B show front and back view of illustrative exosuit 200 according to an embodiment. Exosuit 200 may embody some or all of the base layer, stability layer, power layer, sensor and controls layer, a covering layer, and user interface/user experience (UI/UX) layer, as discussed above. In addition, exosuit 200 may represent one of many different specification implementations of the exosuit shown in FIGS. 1A-1F. Exosuit 200 can include base layer 210 with thigh LDMs 212 and 214, arm LDMs 216 and 218, and upper torso LDM 202. Thigh LDMs 212 and 214 may wrap around the thigh region of the human, and arm LDMs 216 and 218 may wrap around arm region (including the elbow) of the human. Upper torso LDM 220 may wrap around the torso and neck of the human as shown. In particular, LDM 220 may cross near the abdomen, abut the sacrum, cover a portion of the back, and extend around the neck.

Exosuit 200 can include extensor PLSs 230 and 235 secured to thigh LDM 212 and 214 and upper torso LDM 220. Extensor PLSs 230 and 235 may provide leg muscle extensor movements. Extensor PLS 230 may include flexdrive subsystem 231, twisted string 232, and power/communication lines 233. Flexdrive subsystem 231 may include a motor, sensors, a battery, communications circuitry, and/or control circuitry. Twisted string 232 may be attached to flexdrive subsystem 231 and an attachment point 234 on LDM 220. Power/communications lines 233 may convey control signals and/or power to flexdrive subsystem 231. Extensor PLS 235 may include flexdrive subsystem 236, twisted string 237, and power/communication lines 238. Twisted string 237 may be attached to flexdrive subsystem 236 and attachment point 239.

Exosuit 200 can include flexor PLSs 240 and 245 and extensor PLSs 250 and 255 that are secured to LDMs 216, 218, and 220 (as shown). Flexor PLSs 240 and 245 may provide arm muscle flexor movements, and extensor PLSs 250 and 255 may provide arm muscle extensor movements. Flexor PLS 240 may include flexdrive subsystem 241, twisted string 242, and power/communication lines 243. Twisted string 242 may be attached to flexdrive subsystem 241 and attachment point 244. Power/communication lines 243 may be coupled to power and communications module 270. Flexor PLS 245 may include flexdrive subsystem 246, twisted string 247, and power/communication lines 248. Twisted string 247 may be attached to flexdrive subsystem 246 and attachment point 249. Power/communication lines 248 may be coupled to power and communications module 270. Extensor PLS 250 may include flexdrive subsystem 251, twisted string 252, and power/communication lines 253. Twisted string 252 may be attached to flexdrive subsystem 251 and attachment point 254. Power/communication lines 253 may be coupled to power and communications module 270. Extensor PLS 250 may include flexdrive subsystem 256, twisted string 257, and power/communication lines 258. Twisted string 256 may be attached to flexdrive subsystem 256 and attachment point 259. Power/communication lines 258 may be coupled to power and communications module 270.

Exosuit 200 can include flexor PLS 260 and 265 that are secured to thigh LDMs 212 and 214 and LDM 220. Flexor PLSs 260 and 265 may provide leg muscle flexor ARA movements. Flexor PLS 260 may include flexdrive subsystem 261, twisted string 262, and power/communication lines 263. Twisted string 262 may be attached to flexdrive subsystem 261 and attachment point 264. Power/communication lines 263 may be coupled to power and communications module 275. Flexor PLS 266 may include flexdrive subsystem 266, twisted string 267, and power/communication lines 268. Twisted string 267 may be attached to flexdrive subsystem 266 and attachment point 269. Power/communication lines 263 may be coupled to power and communications module 275

Exosuit 200 is designed to assist, resist, and align movements being performed by the user of the suit. Exosuit 200 may include many sensors in various locations to provide data required by control circuitry to provide such movements. These sensors may be located anywhere on base layer 210 and be electrically coupled to power and communications lines (e.g., 233, 237, 243, 247, 253, 257, 263, 267, or other lines). The sensors may provide absolute position data, relative position data, accelerometer data, gyroscopic data, inertial moment data, strain gauge data, resistance data, or any other suitable data.

Exosuit 200 may include user interface 280 that enables the user to control the exosuit. For example, user interface 280 can include several buttons or a touch screen interface. User interface 280 may also include a microphone to receive user spoken commands. User interface 280 may also include a speaker that can be used to playback voice recordings. Other user interface element such as buzzers (e.g., vibrating elements) may be strategically positioned around exosuit 200.

Exosuit 200 can include communications circuitry such as that contained in power and communications module 270 or 275 to communicate directly with a user device (e.g., a smartphone) or with the user device via a central sever. The user may use the user device to select one or more movements he or she would like to perform, and upon selection of the one or more movements, exosuit 200 can the assist, resist, or align movement. The user device or exosuit 200 may provide real-time alignment guidance as to the user's performance of the movement, and exosuit 200 may provide resistance, alignment, or assistance to the movement.

An exosuit can be operated by electronic controllers disposed on or within the exosuit or in wireless or wired communication with the exosuit. The electronic controllers can be configured in a variety of ways to operate the exosuit and to enable functions of the exosuit. The electronic controllers can access and execute computer-readable programs that are stored in elements of the exosuit or in other systems that are in direct or indirect communications with the exosuit. The computer-readable programs can describe methods for operating the exosuit or can describe other operations relating to a exosuit or to a wearer of a exosuit.

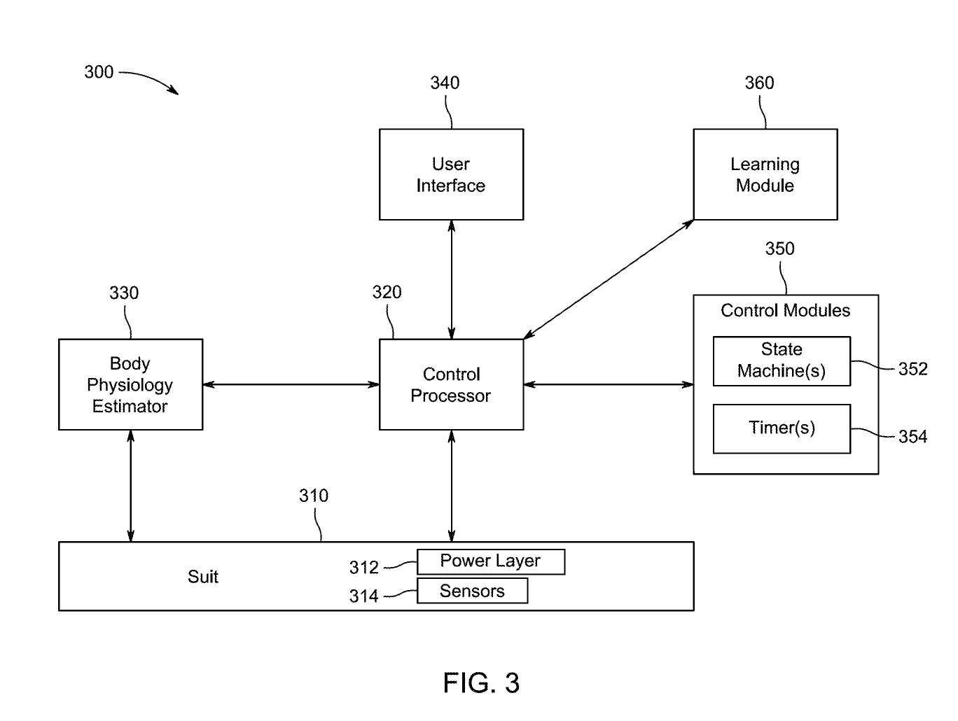

FIG. 3 shows an illustrative symbiosis exosuit system 300 according to an embodiment. The symbiosis enables the exosuit to serve as an autonomous exosuit nervous system that mimics or emulates the nervous system of a lifeform such as a human being. That is, a nervous system is responsible for basic life functions (e.g., breathing, converting food into energy, and maintaining muscle balance) that are performed automatically without requiring conscious thought or input. The autonomous exosuit nervous system enables the exosuit to automatically provide assistance to the user when and where the user needs it without requiring intervention by the user. Exosuit system 300 can do this by tracking the user's body physiology and automatically controlling the suit to provide the anticipated or required support and/or assistance. For example, if a user has been standing for a prolonged period of time, one or more of the muscles being used to help the user stand may begin to tire, and a result, the user's body may exhibit signs of fatigue. Exosuit 300 can observe this muscle fatigue (e.g., due to observed physiological signs) and can automatically cause exosuit 300 to engage the appropriate power layers to compensate for the muscle fatigue.

Symbiosis of exosuit 300 may be expressed in different autonomy levels, where each autonomy level represents a degree to which physiological factors are observed and a degree to which suit assistance or movement actions are performed based on the observed physiological factors. For example, the symbiosis levels can range from a zero level of autonomy to absolute full level of autonomy, with one or more intermediate levels of autonomy. As metaphorical example, autonomous cars operate according to different levels, where each level represents a different ability for the car to self-drive. The symbiosis levels of exosuit operation can be stratified in a similar manner. In a zero level of autonomy, exosuit 300 may not monitor for any physiological cues, nor automatically engage any suit assistance or movement actions. Thus, in a zero level, the user may be required to provide user input to instruct the suit to perform a desired movement or assistance. In an absolute full level of autonomy, exosuit 300 may be able to observe and accurately analyze the observed physiological data (e.g., with 99 percent accuracy or more) and automatically execute the suit assistance or movement actions in a way expressly desired by the user. Thus, in the absolute full level, the exosuit seamlessly serves as an extension of the user's nervous system by automatically determining what the user needs and providing it.

The one or more intermediate levels of autonomy provide different observable physiological results that are accurate but do not represent the absolute nature of the absolute full level of autonomy. For example, the intermediate levels may represent that the exosuit is fully capable of autonomously performing certain actions (e.g., sit to stand) but not others. A corollary to this is ABS braking; the ABS braking system automatically figures out how best to stop the vehicle without requiring the user to pump the brakes or engage in any other activity other than stepping on the brake pedal. In the exosuit context, the exosuit knows when the user wishes to stand from a sitting position, the exosuit knows when the user wishes to perform the movement and engages the appropriate power layer segments to assist in the movement. The intermediate levels may also exist while the exosuit is learning about its user. Each user is different, and the physiological responses are therefore different and particular to each user. Therefore, the ability to discern the physiological cues and the assistance and movements made in response thereto may endure a learning curve before the suit is able to operate at the absolute full level.

FIG. 3 shows that exosuit system 300 can include suit 310, control processor 320, body physiology estimator 330, user interface 340, control modules 350, and learning module 360. Suit 310 can be any suitable exosuit (e.g., exosuit 200) and can include, among other things, power layer 312 and sensors 314. Control processor 320 may process instructions, pass data, and control the suit. Control processor 320 may be connected to suit 310, body physiology estimator 330, user interface 340, control modules 350, and learning module 360. Control processor 320 may provide signals to suit 310 to control, for example, operation of power layer 312.

Body physiology estimator 330 may receive data inputs from sensor 314, control processor 320, and other components if desired. Estimator 330 is operative to analyze the data to ascertain the physiology of the user. Estimator 330 may apply data analytics and statistics to the data to resolve physiological conditions of the user's body. For example, estimator 330 can determine whether the user is sitting, standing, leaning, laying down, laying down on a side, walking, running, jumping, performing exercise movements, playing sports, reaching, holding an object or objects, or performing any other static or active physiological event. The results may be provided to control modules 350, for example, via control processor 320.

Sensors 314 can include an accelerometer, gyroscope, magnetometer, altimeter sensor, EKG sensor, and any other suitable sensor. Sensors 314 may be integrated anywhere within the exosuit, though certain locations may be more preferred than others. The sensor can be placed near the waist, upper body, shoes, thigh, arms, wrists or head. In some embodiments, sensors can be embedded onto the equipment being used by the user. In some embodiments, the sensors can be contained external to the exosuit. For example, if worn on the wrist or arm of a worker, the device can be embedded into a watch, wrist band, elbow sleeve, or arm band. A 20 second device may be used and clipped on the waist on the pelvis, or slipped into a pocket in the garment, embedded into the garment itself, back-brace, belt, hard hat, protective glasses or other personal protective equipment the worker is wearing. The device can also be an adhesive patch worn on the skin. Other form factors can also clip onto the shoe or embedded into a pair of socks or the shoe itself.

Control modules 350 can include various state machines 352 and timers 354 operative to control operation of suit 310 based on outputs supplied by estimator 330, inputs received via user interface 340, and signals provided by control processor 320. Multiple state machines 352 may control operation of the suit. For example, a master state machine may be supported by multiple slave state machines. The slave state machines may be executed in response to a call from the master state machine. In addition, the slave state machines may execute specific assistance functions or movements. For example, each of a sit-to-stand assistance movement, stand-to sit movement, stretch movement, standing movement, walking movement, running movement, jumping movement, crouch movement, specific exercise movement, or any other movement may have its own slave state machine to control suit operation.

Learning module 360 may be operative to learn preferences, peculiarities, or other unique features of a particular user and feedback the learnings to body physiology estimator 330 and control module 350. In some embodiments, learning module 360 may use data analytics to learn about the user. For example, learning module 360 may learn that a particular user walks with a particular gait and cadence. The gait and cadence learnings can be used to modify state machines 352 that control walking for that user. In another embodiment, learning module 360 may incorporate user feedback received via user interface 340. For example, a user may go through an initial setup process whereby the user is instructed to perform a battery of movements and provide responses thereto so that state machines 352 and timers 354 are set to operate in accordance with the preferences of the user.

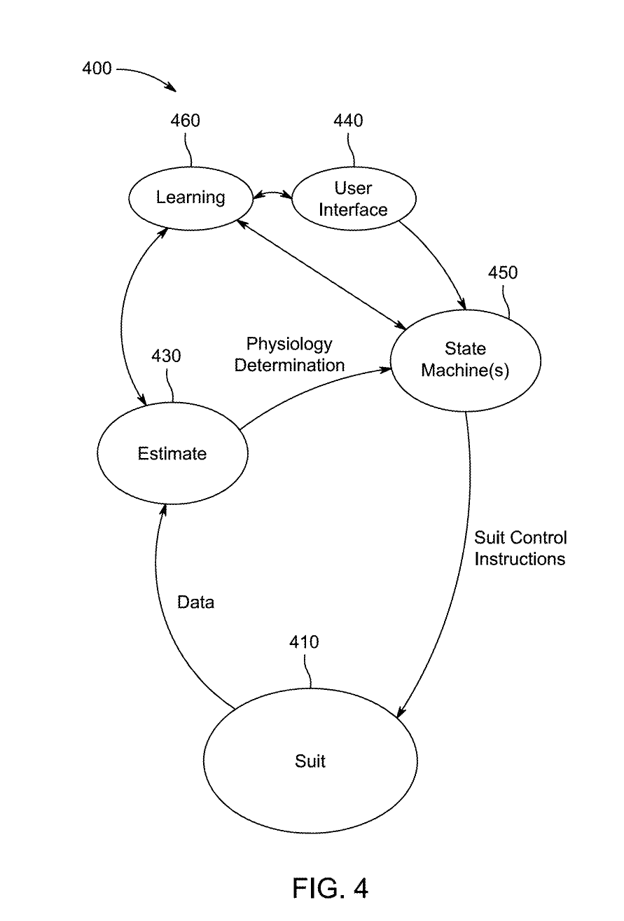

FIG. 4 shows illustrative process 400 for implementing symbiosis exosuit system 300 according to an embodiment. Process 400 includes suite 410, estimator 430, user interface 440, and state machines 450. Process 400 can be represented by a continuous feedback loop in which data is supplied from suit 410 to estimator 430, which provides a physiology determination to state machines 450, which uses the determination to generate suit control instructions that are provided to suit 410. User inputs received via user interface 440 may provide user specified controls that can instruct state machines 450 to execute a particular movement. The autonomous exosuit nervous system is implemented through the continuous feedback loop. The continuous feedback loop enables the autonomous exosuit nervous system to provide rapid response and control of exosuit 410. For example, if the user is sitting down, the estimator 430 can determine that the sitting position is the current physiological determination. Assume that the user reaches for something on a table. Such a movement may result in a movement that appears to be a sit-to-stand. In response to this movement, estimator 430 may register it as the start of a sit-to-stand physiological determination and instruct state machines 450 to initiate a sit-to-stand movement. This way, regardless of whether the user actually stands or sits back down, suit 410 is primed and ready to immediately perform the assistance movement. Further assume that the user sits back down (after having grabbed the item on the table). In response to initiation of the sit down movement, estimator 430 can make this determination as it is happening and instruct state machines 450 to cease the sit-to-stand operation. Thus, the continuous feedback loop provides real-time assessment and instantaneous suit controls in response to the user's immediate physiological needs, and not after.

In some embodiments, estimator 430 may be able to determine that the user is was attempting to reach something on the table while also performing the motion that includes at least the start of a sit to stand movement. Estimator 430 may be able to correlate the reaching motion with the sit-to-stand motion and decide that the user does not actually need to stand, but may require an appropriate amount of assist to reach the item. In this particular situation, state machine 450 may activate a power layer segment (e.g., a particular one of the hip extensors) to provide the user with the reach assistance.

Learning 460 can receive and provide data to estimator 430, user interface 440, and state machines 450. Learning 460 may be leveraged to update state machines 450 and/or estimator 430.

Embodiments discussed herein refer to using exosuits to monitor worker safety and performance. That is, some applications of exosuits may be used in industrial applications in which workers don and use the exosuit to perform their duties. For example, in one embodiment, workers may use exosuits to perform heavy lifting tasks or to operate heavy equipment. The exosuit can monitor the workers as they perform their duties. The monitoring can enable injury detection or injury prevention. For example, the sensor data may show that the worker is exhibiting signs of fatigue or improper form in movement, both of which may lead to injury. The exosuit can provide additional assistance to compensate for the fatigue or improper form, or can provide feedback to inform the worker of the same. In other embodiments, the exosuit may be used to monitor worker productivity. If desired, an aggregate of work productivity data may be collected to assess worker productivity.

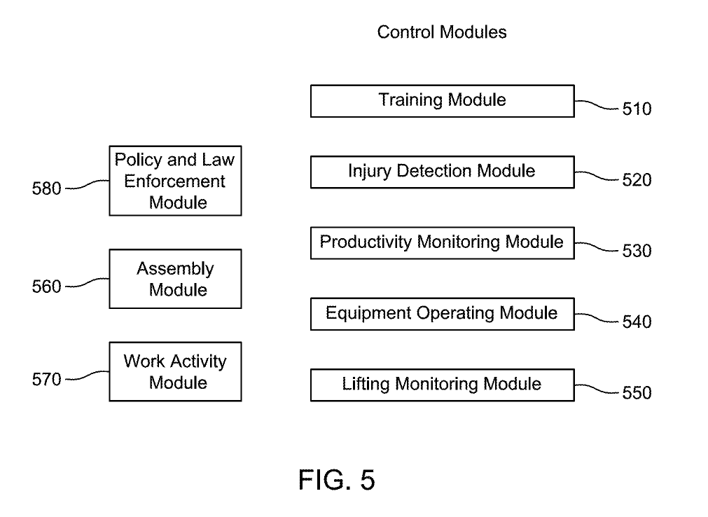

FIG. 5 shows an illustrative diagram of different control modules that may be implemented by an exosuit according to an embodiment. For example, the control modules of FIG. 5 may be implemented in control module 350 of FIG. 3. FIG. 5 can include training module 510, injury detection module 520, productivity monitoring module 530, equipment operating module 540, lifting module 550, assembly module 560, work activity module 570, and policy and law enforcement module 580. Other modules may be added as required. Each of the modules in FIG. 5 may be specifically configured to operate in connection with the suit by monitoring physiological movement of the user of the exosuit (e.g., via one or more sensors existing on the exosuit), controlling operation of the power layers of the exosuit, providing feedback to the user via the exosuit itself or by transmitting data to a device (e.g., a personal device) capable of providing the feedback.

Training module 510 may be accessed to provide on-the-job training of movements required to be performed by the worker during his or her shift. The training movements can include, for example, heavy lifting movements or ordinary lifting movements, heavy equipment operational movements, or conventional equipment operational movements, assembly movements, and any other suitable movement that can benefit from the use of an exosuit. The training module may include a step-by-by instruction course on how to use the exosuit in connection with the movement. The exosuit can be used to train individuals on how to properly operate the equipment, walking a user through all safety features and providing real-time feedback on form and technique. Feedback can include the orientation and position of the user's forearm, upper-arm, shoulders, head, neck, pelvis and feet. The training can also include providing real-time guidance on the proper orientation and use of equipment itself. For example, the training can provide feedback that educates workers how to pick up objects properly with good bending techniques.

Injury detection module 520 may be used to detect injury in a worker or to detect conditions that may lead to injury. Injury detection module 520 may have the ability to detect injuries that may occur on a relatively short term basis or a relatively long term basis. Relatively short term injuries may be those that occur as a result of a worker movement that causes an immediate step change in body physiology (e.g., such as pulled muscle or broken bone). Relatively long term injuries may be those that occur over a longer period of time (e.g., a result of repetitive motion). Injury detection module 520 can detect job specific injuries. As a specific example, injury detection module 520 can detect injuries that are commonly found with construction workers. Construction workers often work with equipment that exposes the worker to large forces on the human body. High intensity and high frequency vibrations from operating jack hammers, vibrating hand-held tools, saws and other equipment can have significant long-term health impacts such as Vibration Syndrome, vibration-induced white finger, and carpal tunnel syndrome. Vibration Syndrome can refer to a group of symptoms relating to the use of vibrating tools. Examples of Vibration Syndrome can include muscle weakness, fatigue, pain in the arms and shoulders, and blood-circulation failures in the fingers leading to a condition known as white finger.

Injury detection module 520 may make injury assessments using many different approaches. For example, in one approach, injury detection can done by analyzing the before and after walking gait of the user. There are predominant differences in walking gait between a healthy person and the same injured person. For example, the exosuit can detect distinct indicators of injury such as increased left and right gait asymmetry, increased pelvic rotation ranges, increased lateral sway, and sharp decreases in overall movement activities. Injury detection module 520 may characterize the injury event by detecting if a fall occurred or if a user was operating the equipment improperly or if the user was fatigued

Injury detection module 520 may provide an alert if an injury has been detected. For example, if a fall has been detected and the worker is immobile, the exosuit can flash bright red and white LED lights and blasts a message such as “Help, Man Down” over the exosuit's audio speaker. The exosuit can also triangulate and communicate the location of the fallen worker via GPS or Wi-Fi or leveraging other indoor tracking technologies. The exosuit may also trigger other lights or safety mechanisms the worker is wearing or carrying. For example, the exosuit can cause remote device such as a connected head lamp to flash pulses of light, or it can connect to the user's smartphone or handheld radio to broadcast a message to help rescue teams locate the person. Emergency alerts can be sent out to a team lead or safety lead if a worker is determined to be injured. For example, the closest teammate can also be alerted to assist the worker. Emergency Medical Service (EMS) can be configured to be notified automatically. In some embodiment, the user may cause an emergency alert to be sent out in the event that the exosuit system does not automatically detect the injury.

Productivity monitoring module 530 may monitor the productivity of one or more workers. For example, module 530 can quantify the amount of time the user is working on the job and how effective the worker is at doing his job. By monitoring the movement activities, vibrational intensities and arm motion activities, the exosuit can determine how productive an individual worker is compared to peers. Module 530 can identify workers who are over or underperforming. In some embodiments, over performing workers may be provided with micro bonuses (e.g., a free lunch) or over time, the consistent determination of over performance may be rewarded with a monetary year-end bonus. Module 530 can also monitor performance of the worker in completing tasks such as, for example, assembly speed and loading speed. Module 530 can ensure that the worker is conforming to mandatory work breaks, lunch breaks, etc. to ensure compliance with employment laws. Module 530 can be used as a motivational tool to encourage workers to increase their productivity. For example, module 530 may present competition like events among the workers to increase productivity and improve morale. Module 530 can determine which workers are at high risk of injury or are being particularly unproductive, and send an alert to a central system, worker or safety manager. In some embodiments, module 530 can automatically find another individual who is well rested who can take the place of the worker determined to be underperforming or at risk of injury to keep the job going.

Productivity monitoring module 530 may be able to coordinate collection of data from a multitude of source and interpret that data to process worker productivity. For example, data may be collected from the exosuit, security cameras, badge stations, and other equipment. This way data across the entire workforce and workplace can be leveraged to optimize the productivity and safety of the workers. Predictive machine intelligence algorithms can be used to identify abnormal activities that correlate with injury and identify new red flag predictors that translate all the way back down to each individual. Machine intelligence models can also be used to predict worker productivity and identify opportunities for process and worker improvement. For example, a machine intelligence or algorithmic logic model can be used to measure the gradual decreases in worker productivity, increases in sedentary behavior, and predict worker attrition or injury.

A web dashboard can provide the safety manager or site manager with information on the productivity of the entire task force, identify which workers are at risk of injury from vibration overexposure and which workers are under performing. Under performing workers can be identified by the lack of movement, steps, arm swings, high impact or vibration exposure. The sensors can detect when a user is sitting or even lying down, thus if a worker plans to sleep on the job, the system can alert the manager.

The data may be used to identify issues with equipment being used by the workers. For example, the data can identify which equipment transmits the largest vibration intensities and may therefore need repair or replacement. The number of fall detections or injuries can be geolocated to identify if there are certain high-risk areas in the construction site that needs to be addressed or give worker more caution.

Equipment operating module 540 may monitor a worker's use of equipment to ensure that the worker is using the equipment properly. The equipment can vary in size and complexity, including for example, heavy equipment such as construction equipment, mining equipment, and other “heavy” equipment and lite equipment such as assembly line equipment, lawn and garden equipment, or other “lite” equipment. Module 540 can provide analysis of proper ergonomic biomechanics for equipment operation before, during and after operation. The exosuit sensors can measure the orientation angles relative to the ground. Orientation angle along with equipment location and use can be used to approximate whether the user is operating the equipment ergonomically. For example, when the equipment is in operation, the orientation can change instantaneously due to vibrational forces being exerted on to the worker. Module 540 can quantify the orientation during operation.

Furthermore, while in operation, module 540 can calculate the changes in displacement (lateral, forward/backward and up/down). While some displacement is expected, namely in the vertical and forward/backward planes, significant changes in displacement can be an indicator of instability, especially in directions (such as lateral) where little displacement is expected. This may mean the worker is fatigued, has poor biomechanics during operation, or both. For example, when a user is about to operate equipment, such as a jackhammer, the arms should be held at specific angles relative to the ground to maintain proper control of the jackhammer throughout operation. If significant changes in arm orientation are detected, the worker may be losing control of the jack hammer, or is losing grip of the handles, whereas if the orientation is stable, the worker has proper control. Furthermore, if there is significant lateral displacement detected, it again shows that the user is losing control or is using improper biomechanics. When events like this occur, module 540 can record and notify the worker immediately to address the issue.

Module 540 can prompt the worker to stretch or perform certain exercises before and after equipment operation to help mitigate and avoid injury. During such a prompt, module 540 can record and measure the stretches and exercises the worker actually did to help measure compliance.

Module 540 can leverage data obtained from sensors placed on the equipment in conjunction with or to the exclusion of sensors in the exosuit The equipment sensors can, for example, calculate the orientation (and orientation variability) of the equipment during operation and quantify the displacement (and displacement variability). If the equipment is operated with improper orientation, or has unexpected changes in orientation, or significant displacements in a direction that should not occur, the device can send feedback to the worker or site managers. Module 540 can corroborate exosuit data with equipment data to determine whether the worker is using the equipment properly.

Lifting module 550 may control the exosuit to assist the worker in performing lifting operations. For example, the worker may be required to perform a series of lifting moves as part of his shift. The exosuit, in combination with lifting module 550, can assist the worker in performing those moves. For example, if the worker is required to lift and place object, the exosuit can assist in those movements. In some embodiments, the weight of the objects may be too heavy for the worker to lift without exosuit assistance.

Assembly module 560 may control the exosuit to assist the worker in performing tasks associated with assembly of an object. In one embodiment, the assembly can include construction of the object from start to finish. In another embodiment, the assembly can be a stage in an assembly line process.

Work activity module 570 may control the exosuit to assist the worker in performing any suitable worked related tasks. Module 570 may represent a catch all module for controlling the exosuit in any manner deemed suitable for the worker's job requirements.

Policy and law enforcement module 580 may be used to ensure that workers are complying with company policies and the law. For example, some companies may have policies that govern the safety and expectations of its workers. Workers wearing the exosuit can be monitored to ensure that those policies are followed. In addition, worker compensation laws, employment laws, and other laws or regulations (e.g. OSHA) promulgated by governing bodies require strict compliance. Exosuits can monitor the worker to ensure that the relevant laws and regulations are being abided.

In order for the control modules (e.g., modules shown in FIGS. 3 and 5) to perform their respective tasks, the control modules require knowledge of the physiology or bio-mechanical movements of the worker wearing the exosuit. As indicated in FIG. 3, sensors 314 may provide movement data to body physiology estimator 330, which may analyze the data to extrapolate physiological or bio-mechanical movements of the worker. When the movements of the worker are known, control modules can use this information to provide worker safety monitoring, worker productivity monitoring, exosuit based worker assistance, and compliance monitoring according to various embodiments.

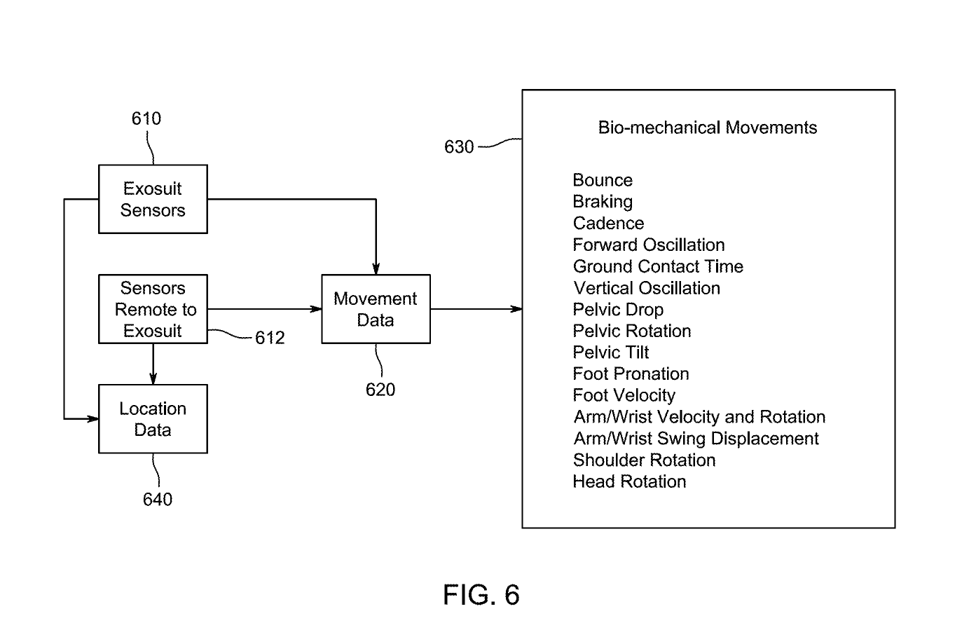

FIG. 6 shows an illustrative block diagram according to an embodiment. In particular, FIG. 6 shows that exosuit sensors 610 provide movement data 620, which can be extrapolated to bio-mechanical movements 630. Sensors remote to the exosuit 612 may also provide movement data. For example, sensors remote the exosuit may include sensors residing on a piece of equipment being used by the worker. An illustrative, and non-exhaustive, list of bio-mechanical movements 630 is shown. For example, bio-mechanical movements 630 can include bounce, braking, cadence, forward oscillation, vertical oscillation, ground contact time, pelvic drop, pelvic rotation, pelvic tilt, foot pronation, foot velocity, arm or wrist velocity, arm or wrist rotation, arm or wrist swing displacement, shoulder rotation, and head rotation. Bio-mechanical movements 630 can represent various components of generic body movements such as, for example, walking, standing, running, squatting, lifting, throwing, etc. Categorizing generic movements into granular bio-mechanical movements provides a rich data set for accurately detecting and monitoring many or all aspects of the generic movement. Such data may enable the control modules to execute their programming with a high degree of accuracy and effectiveness. The movement data 620 may be used obtain other metrics such as overall steps, energy expenditure, duration and intensity of exposures to vibrational activities, duration and biomechanics of proper operation of equipment, overall activity of hands, lack of sedentary behavior, and peak impact analysis.

The sensor data can be used to determine, for example, the time spent walking, running, sitting, standing, or lying down, the number of walking steps or running steps, the number of calories that were burned during any activity, the posture of a user during any of activity, and body stretches, range of motion arm and leg swings and various exercises. Additionally, while walking or running, the sensor data can be used quantify gait dynamics such as pelvic stability, range of motion in degrees of pelvic drop, tilt and rotation, the amount of vertical oscillation of the pelvis, forward/backward braking forces, step cadence (number of steps per minute), stride asymmetry, ground contact time, left step/right step, turning velocity, peak velocity, and limp detection. The sensor data can be used to detect shock events and vibration events. The sensor data can be used to detect lifting characteristics such as, for example, proper lifting from the knees, improper lifting from the lower back, twisting, and bending from the waist.

The sensor data may be used to validate whether a worker was injured on the job, measure worker productivity and identify if a worker is at risk of injury due to a newly detected limp, balance and sway characteristics in walking gait. Worker productivity is yet another metric that can be measured by a number of other activities including overall steps, energy expenditure duration and intensity of exposures to vibrational activities, duration and biomechanics of proper operation of equipment, overall activity of hands, lack of sedentary behavior, and peak impact analysis. For example, the sensor data may indicate a sudden abnormal walking gait from a worker with increased lateral sway and left/right leg stance time asymmetry—indicators of walk instability, potentially due to a sudden injury. In another case, the sensor data can indicate when a worker is carrying too much weight by detecting a bending motion followed by increased lateral sway and pelvic rotation.

FIG. 6 also shows that exosuit sensors 610 and sensors remote to exosuit 612 can provide location data 640. Location data 640 can indicate the location of the worker within the workplace, such as a particular room or section of a store, factory, work site, mine, etc. As such, location data 640 may include GPS data, wireless signals (e.g., Wi-Fi, Cellular, Bluetooth, ZigBee) for establishing location via triangulation, camera data that shows location of the user, transponder data, badge in/badge out data, or any other data that discloses the location of the worker. Location data may be used by one or more of the control modules. For example, module 580 may use location data to determine whether the worker is taking a mandatory break. As another example, a module may use the location data to activate certain exosuit features based on the location of the worker.

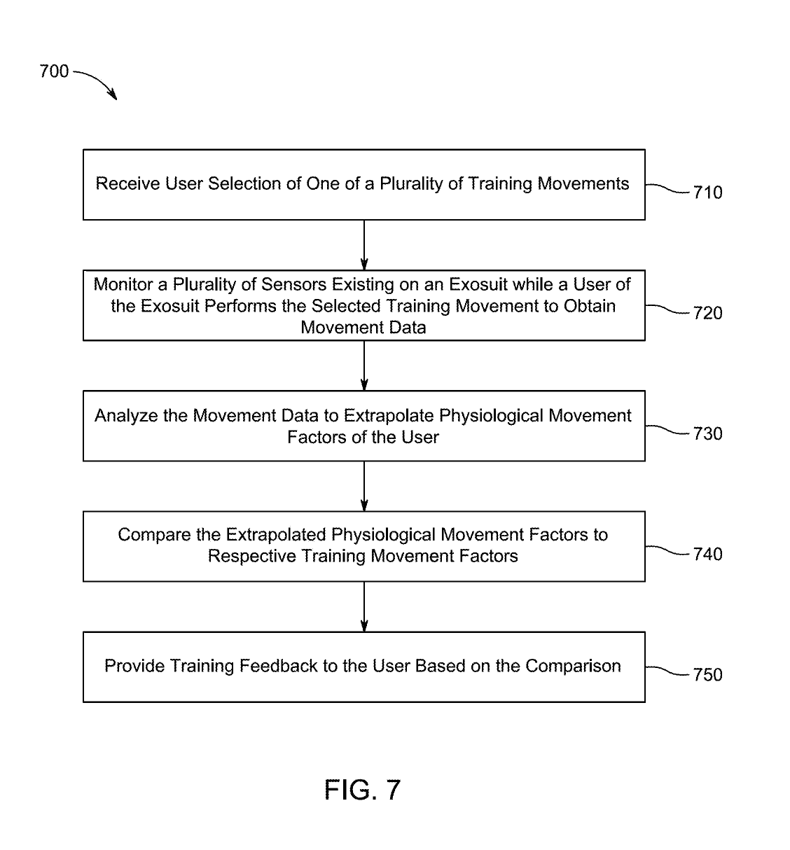

FIG. 7 shows illustrative training process 700 according to an embodiment. Process 700 may be implemented by training module 510 of FIG. 5 and further implemented while a worker is wearing an exosuit. Process 700 may begin at step 710, by receiving user selection of one of a plurality of training movements. Training movements may be work-related movements are that performed by a worker during the course of his or her work day. For example, the work-related movements may be any of those implemented by other control modules according to embodiments discussed herein. One or more sensors, existing on an exosuit, may be monitored while a user of the exosuit performs the selected training movement to obtain movement data, as indicated in step 720. The sensors can be sensors 314, for example. If desired, additional sensors that are not integrated with the exosuit may be used to provide movement data. At step 730, the movement data are analyzed to extrapolate physiological movement factors or bio-mechanical movements (e.g., movements 630 of FIG. 6). The physiological movement factors can be same thing as bio-mechanical movements.

At step 740, the extrapolated movement factors can be compared to respective training movement factors. The training movement factors may represent a desired range of values within which the extrapolated movement factors should fall. That is, every movement factor may be expected to fall within a range of values of the corresponding training movement factor. For example, if the comparison indicates that the extrapolated movement factors approximate their respective training movement factors, it may be inferred that the worker is performing the training movement correctly. If the comparison indicates that one or more of the extrapolated movement factors do not adequately approximate their respective training movement factors, it may be inferred that the worker is not performing the training movement correctly. It should be understood that the training movement factors can be tailored to the characteristics (e.g., anatomy) of the worker. For example, a short person may have different training movement factors than a tall person. The appropriate training factors can be selected based on any suitable factors, such as size of the exosuit donned by the worker, a calibration phase implemented by the exosuit, or a questionnaire filled out by the worker prior to training.

At step 750, training feedback can be provided to the worker based on the comparison in step 740. Both positive and constructive feedback can be provided. For example, the comparison indicates that the user is performing the training movement correctly, positive feedback may be provided. If the comparison indicates that the user is not performing the training movement correctly, constructive feedback can be provided. The specificity of the feedback may vary, for example to general feedback (e.g., “you're a doing a great job” or “you are doing it wrong”) to specific feedback (e.g., “your back angle is too steep”). The feedback can be in the form of audio and/or visual cues originating from the exosuit itself (e.g., the exosuit has a speaker, display, or indicator lights) or a device remote to the user use such as a monitor, smart watch or band, or mobile device may provide the cues. The feedback can be provided by the power layers of the exosuit. That is, the one or more power layers may be selectively activated to guide or assist the worker in performing the training move correctly. For example, in addition to the “your back angle is too steep” feedback, PLSs around the upper torso and waist may be activated to provide support to the worker, allowing them to feel the difference between the correct and incorrect movement. In addition to providing biomechanical support, this type of physical feedback provides haptic guidance for workers to train their neuromuscular pathways by learning how the correct movement should feel. In some embodiments, exosuit assisted corrections may only be used when the comparison indicates that the user is not performing the training movement correctly. In some embodiments, the feedback can be directly correlated to one or more movement factors. That is, if a first factor is out of range based on the comparison, the feedback (e.g., voice, display, and/or power segment) may indicate what needs to be adjusted, based on the first factor, to correct the movement.

It should be appreciated that the steps shown in FIG. 7 are merely illustrative and that additional steps may be added.

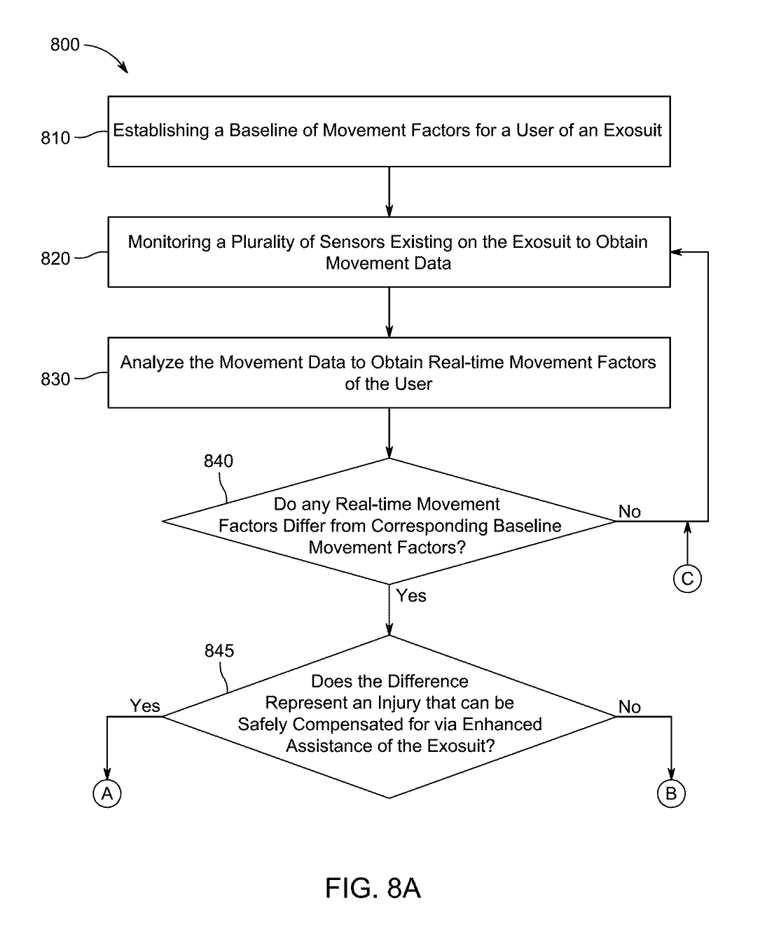

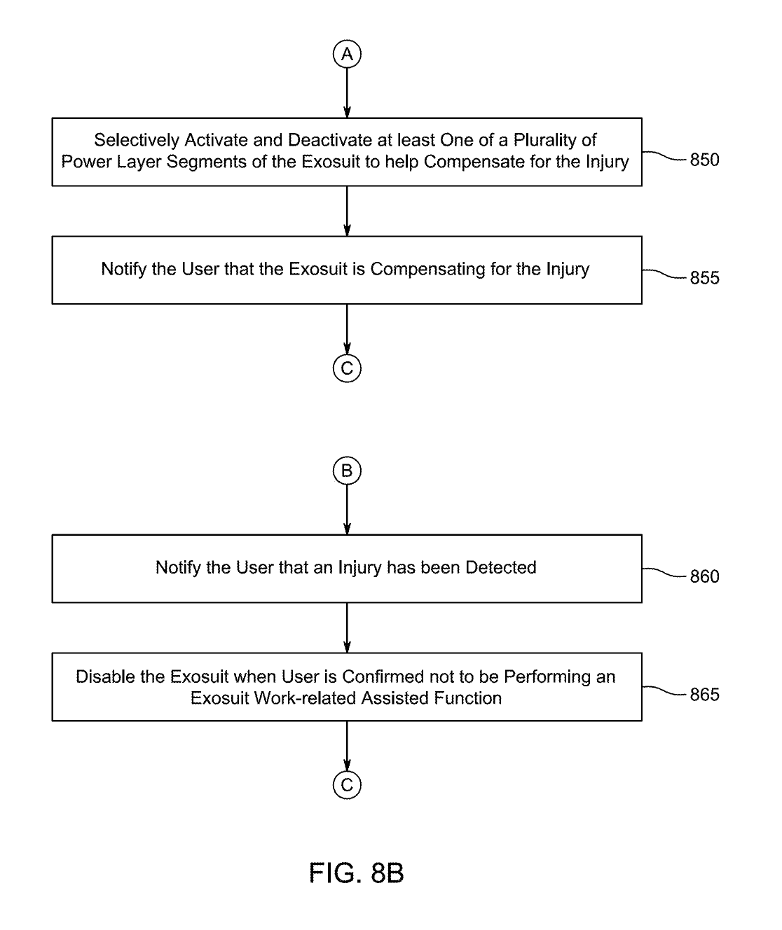

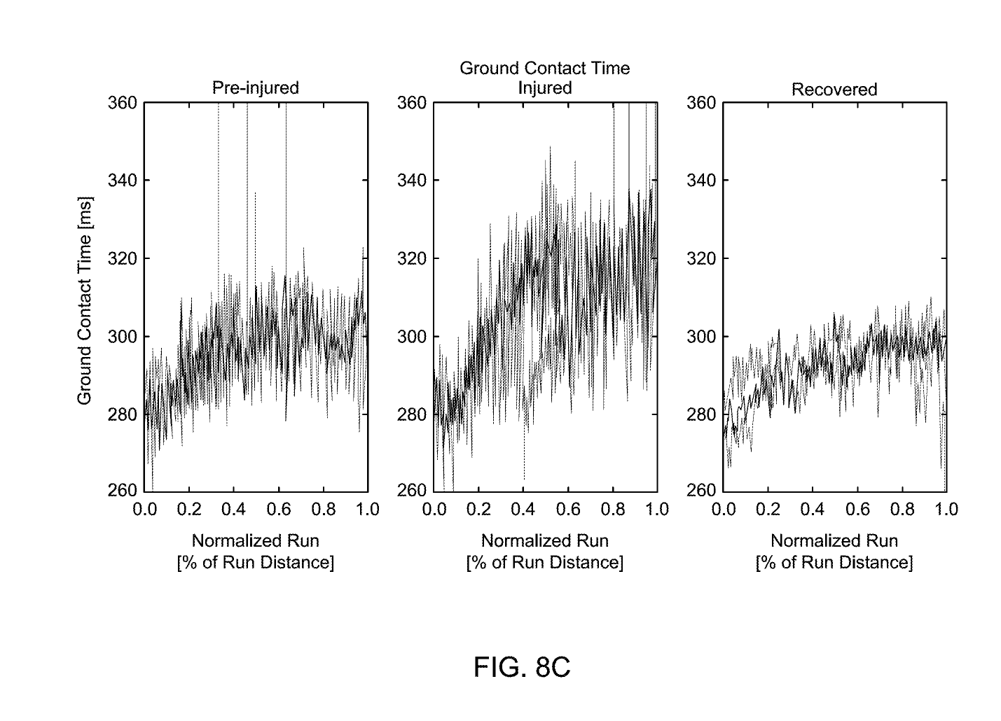

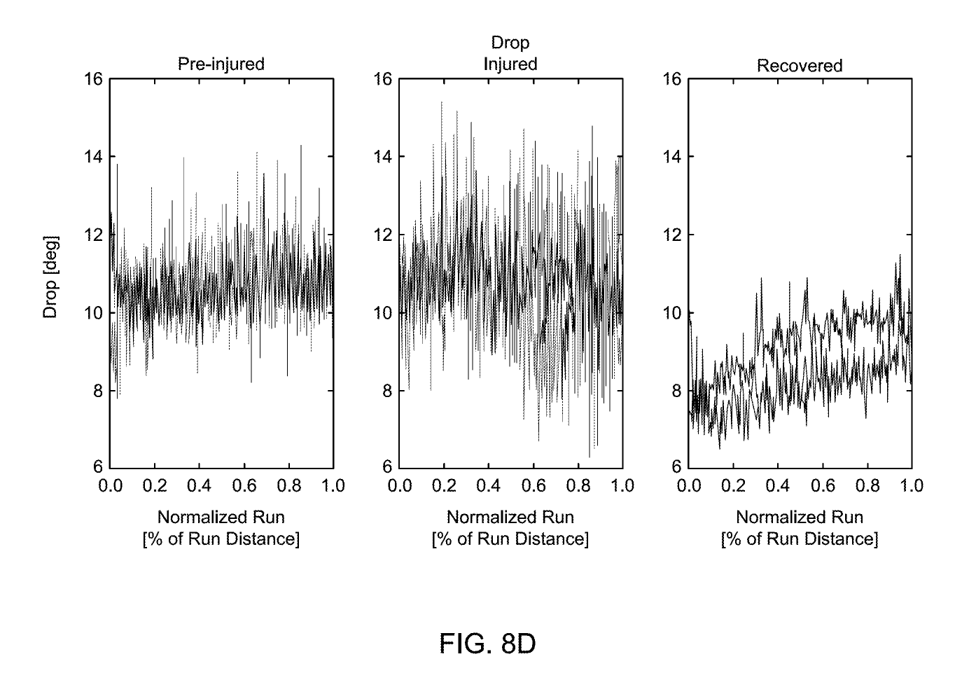

FIGS. 8A and 8B show illustrative injury detection process 800 according to an embodiment. Process 800 may be implemented by injury detection module 520 of FIG. 5 and further implemented while a worker is wearing an exosuit. Process 800 may begin at step 810 by establishing baseline movement factors for a user of an exosuit. The movement factors can be bio-mechanical movement factors. The baseline movement factors can be a historical record of the user's movements. In some embodiments, the baseline movement factors can be an average of the user's movement records. The average can be calculated based on a moving window of sample sizes. It should be appreciated that many different techniques can be used to establish baseline movement factors. The baseline movement factors are used to serve as a starting point (or before injury event) of movement factors that can be compared against real-time or recently acquired movement factors. Any detected differences of movement factors from the baseline may indicate occurrence of an injury or the potential for an onset injury.

FIGS. 8C and 8D shows illustrative graphs of movement data pertaining to a particular person. Both FIG. 8C shows movement data corresponding to pre-injury, injured, and exemplary exosuit correction for an individual with an Iliotibial band syndrome, a repetitive strain injury. FIG. 8C shows ground contact time and FIG. 8D shows pelvic drop. The graphs pertaining to pre-injury and injury show noticeable disparity magnitudes. That is, the injury graphs show a wider range of magnitude compared to the pre-injury graphs. The exemplary exosuit correction graphs show that exosuit can compensate for the injury, as evidenced by the substantial reduction in magnitude as compared to the injured data magnitudes.

One or more sensors existing on the exosuit and/or elsewhere can be monitored to obtain movement data, as indicated by step 820. The movement data can be analyzed to obtain real-time movement factors of the user, as indicated by step 830. If desired, the real-time movement factors can be time averaged across a static or dynamic window of sample sizes. At step 840, a determination is made as to whether any real-time movement factors (or average thereof) differ from corresponding baseline movement factors. The magnitude of the difference may vary depending on the severity of the injury. A slip and fall injury may produce a large magnitude difference, whereas fatigue may produce a relatively small, but persistent, difference. Process 800 has the ability to track these differences and make informed decisions as to the degree of the injury and the potential for injury.

If the determination at step 840 is NO, process 800 may loop back to step 820. If the determination at step 840 is YES, process 800 may determine if the difference represents an injury or potential onset injury that can be safely compensated for via enhanced assistance of the exosuit, as indicated in step 845. If the determination at step 845 is YES, process 800 can selectively activate and deactivate at least one of a plurality of power layer segments of the exosuit to help compensate for the injury or potential onset injury, as indicated in step 850. In one embodiment, the exosuit may increase its assistance above that which it may already be providing to compensate for the injury or to prevent injury. The one more power layer segments being selectively activated may correspond to the real-time movement factors determined to differ from the baseline movement factors. For example, referring to FIGS. 8C and 8D, the exosuit corrected graphs shows that the user is performing just as well, if not better, than the pre-injury graphs. At step 855, the user may be notified that the exosuit is compensating for the injury or the potential onset injury. Following step 855, process 800 may return to step 820.

If the determination at step 845 is NO, process 800 can notify the user that an injury has been detected, as indicated in step 855. At step 865, the exosuit may be disabled when the user is confirmed not to be performing an exosuit work-related assisted function. In some embodiments, step 865 is meant to prevent the worker from continuing to work after an injury has been detected, but if the worker is in the middle of work movement, the exosuit will allow the user to complete the move or otherwise extricate himself from the movement in a safe manner with the assistance of the exosuit. Following step 860, process 800 may proceed to step 820.

It should be appreciated that the steps shown in FIGS. 8A and 8B are merely illustrative and that additional steps may be added.

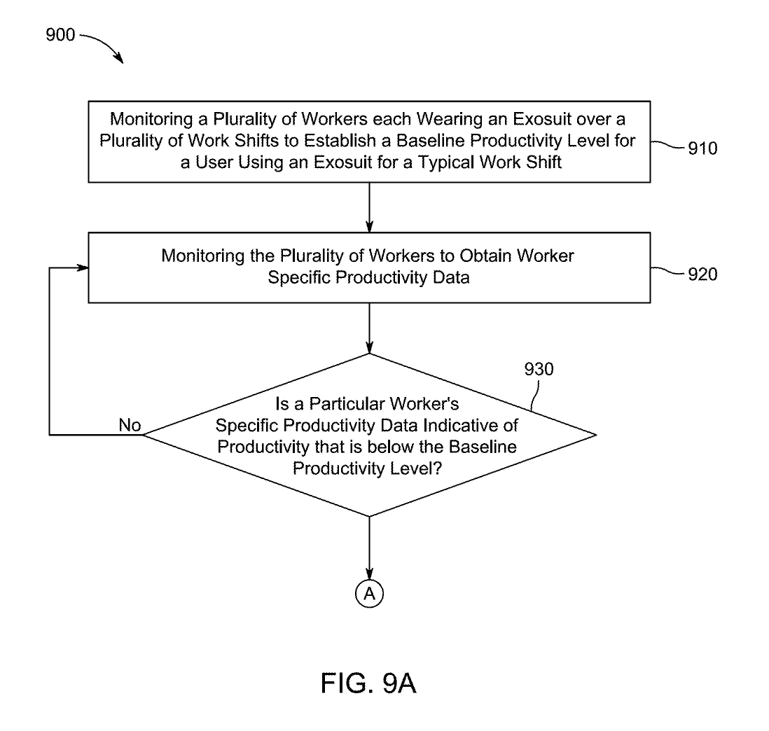

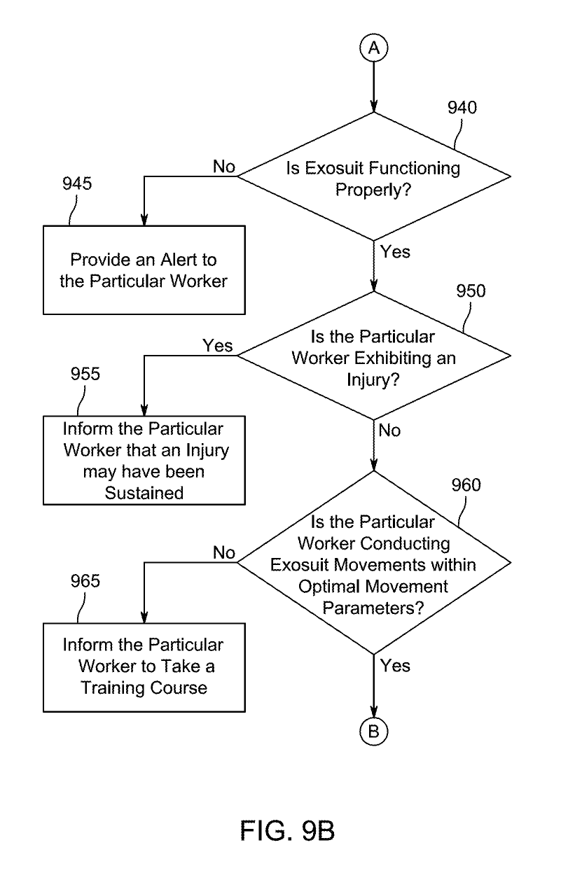

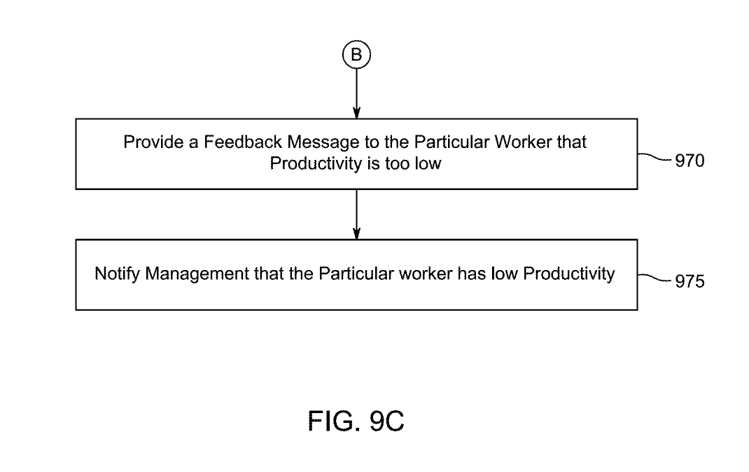

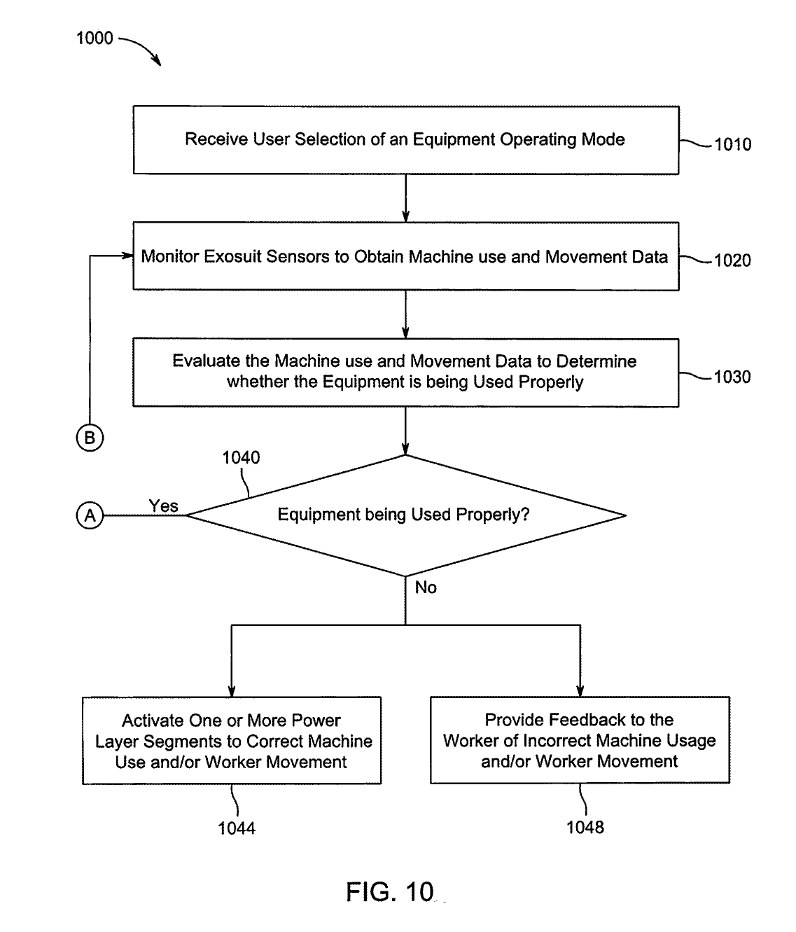

FIGS. 9A-9C show illustrative worker productivity process 900 according to an embodiment. Process 900 may be implemented by productivity monitoring module 530 of FIG. 5 and further implemented while a worker is wearing an exosuit. Process 900 may begin at step 910 by monitoring several workers, each wearing an exosuit, over a plurality of work shifts to establish a baseline productivity level for a user using an exosuit for a typical work shift. The baseline may change over time as the worker population changes. Thus, in some embodiments, the baseline represents a snap shot in time of worker productivity. At step 920, the workers are monitored to obtain worker specific productivity data. That is, metrics can be acquired for each worker over each shift. The worker specific productivity data may include bio-mechanical movement factors, location data (e.g., tracking the location of the worker throughout his shift), widget assembly time (e.g., time it takes for worker to complete a task), widget processing numbers (e.g., the number of widget the worker processed within a given time), and any other productivity factors.

At step 930, a determination is made as to whether a particular worker's specific productivity data is indicative of productivity that is below the baseline productivity level. This determination can be done by comparing worker productivity data to the baseline productivity level. The baseline productivity level can include the same metrics as the worker specific productivity data. Thus, the degree to which comparisons and extrapolations can be made are limitless. If the determination at step 930 is NO, process 900 may loop back to step 920. If the determination is YES, process 900 can proceed to step 940, which determines whether the exosuit is functioning properly. If the exosuit is not operating within normal parameters, this may explain why the worker's productivity has fallen below a baseline. If the determination at step 940 is NO, process 900 may provide an alert to the particular worker, as indicated in step 945. For example, the alert may inform the worker that the battery packs need to be replaced or that one or more of the power layer segments require replacement. If the determination at step 940 is YES, process 900 may proceed to step 950, which can determine if the particular worker is exhibiting an injury.

Determination of an injury can be implemented by a sub-routine such as described in connection with FIG. 8 or injury detection module 520. If the worker is injured, this may be the reason why his productivity has fallen. If the determination at step 950 is YES, process 900 may inform the worker that an injury may have been sustained, as step 955. In some embodiments, steps for handling an injury as described in connection with process 800 may be implemented in lieu of steps 950 and 955. If the determination at step 950 is NO, process 900 can proceed to step 960.

At step 960, a determination is made as to whether the particular worker is conducting exosuit movements with optimal movement parameters. If the determination at step 960 is NO, the particular worker may be instructed to take a training course to re-familiarize himself with the proper movement. In some embodiments, the training process 700 may be conducted if the determination at step 960 is NO. If the determination at step 960 is YES, process 900 may proceed to step 970.

At step 970, a feedback message may be provided to the particular worker that his productivity is too low. At step 975, a management may be notified that the particular worker has low productivity.

It should be appreciated that the steps shown in FIGS. 9A-9C are merely illustrative and that additional steps may be added. For example, steps may be added to detect whether the particular worker is exhibiting fatigue symptoms. If the determination is YES, the worker may be instructed to take a break.

FIGS. 10A-10B show illustrative equipment operating process 1000 according to an embodiment. Process 1000 may be implemented by equipment operating module 540 of FIG. 5 and further implemented while a worker is wearing an exosuit. Process 1000 may begin at step 1010, in which a user selection of an equipment operating mode is received. For example, the user may select to use a relatively heavy piece of equipment that requires substantial physical manipulation to handle. At step 1020, exosuit sensors can be monitored to obtain machine use and movement data. In some embodiments, one or more sensors on the equipment may provide data to complement the machine use and movement data. At step 1030, the machine use and movement data can be evaluated to determine whether the equipment is being used properly. For example, process 1000 can perform analysis of proper ergonomic biomechanics for equipment operation before, during and after operation. Process 1000 can measure the orientation angles relative to the ground. Orientation angle along with device location (whether the device is worn on forearm, upper limb, pelvis, etc.) can be used to approximate whether the user is operating the equipment ergonomically. When the equipment is in operation, the orientation can change instantaneously, for example, due to the vibrational forces. Process 1000 can perform averaging over a period of time to quantify the orientation during operation. Furthermore, process 1000 can calculate the changes in displacement (lateral, forward/backward, and up/down). While some displacement is expected, namely in the vertical and forward/backward planes, significant changes in displacement can be an indicator of instability, especially in directions (such as lateral) where little displacement is expected. This change in measured performance may mean the worker is fatigued, has poor biomechanics during operation, or both.

If the equipment is not being used properly (at step 1040, process 1000 can be activated one or more power layer segments to correct machine use and/or worker movement (step 1044). In addition, feedback may be provided to the worker informing said worker of the incorrect machine usage and/or worker movement (step 1048). For example, when a user is about to operate equipment, such as a jackhammer, the arms need to be held at specific angles relative to the ground to maintain proper control of the jackhammer throughout operation. If significant changes in arm orientation are detected, the worker may be losing control of the jack hammer, or is losing grip of the handles, whereas if the orientation is stable, the use has proper control. Furthermore, if there is significant lateral displacement detected, this may indicate that the user is losing control or is using improper biomechanics. The exosuit can assist in correcting the user and/or notify the worker immediately to address the issue.

The sensor and software system can also prompt the worker to stretch or perform certain exercises before and after equipment operation to help mitigate and avoid injury. During such a prompt, the sensor and software system can record and measure the stretches and exercises the worker actually did to help measure compliance. Additional details on how training movements can analyzed and verified can be found in commonly owned U.S. Patent Publication No. 20180133551, and commonly owned U.S. patent application Ser. No. 16/182,102, filed Nov. 6, 2018, the disclosures of which are incorporated by reference herein.

If the equipment is being used properly (at step 1040), process 1000 may proceed to step 1050. At step 1050, a determination is made whether the worker is fatigued. If YES, process 1000 may provide feedback to the user at step 1055 that, for example, that the user may want to consider taking a break. Alternatively, or in addition to, the feedback may result in extra assistance being provided by the exosuit. If the determination at step 1050 is NO, a determination can be made whether the worker is injured or about to be injured. If the determination at step 1060 is YES, process may provide feedback at step 1055. If the determination at step 1060 is NO, process 1000 may return to step 1020.

It should be appreciated that the steps shown in FIGS. 10A-10B are merely illustrative and that additional steps may be added and the order of the steps may be rearranged.

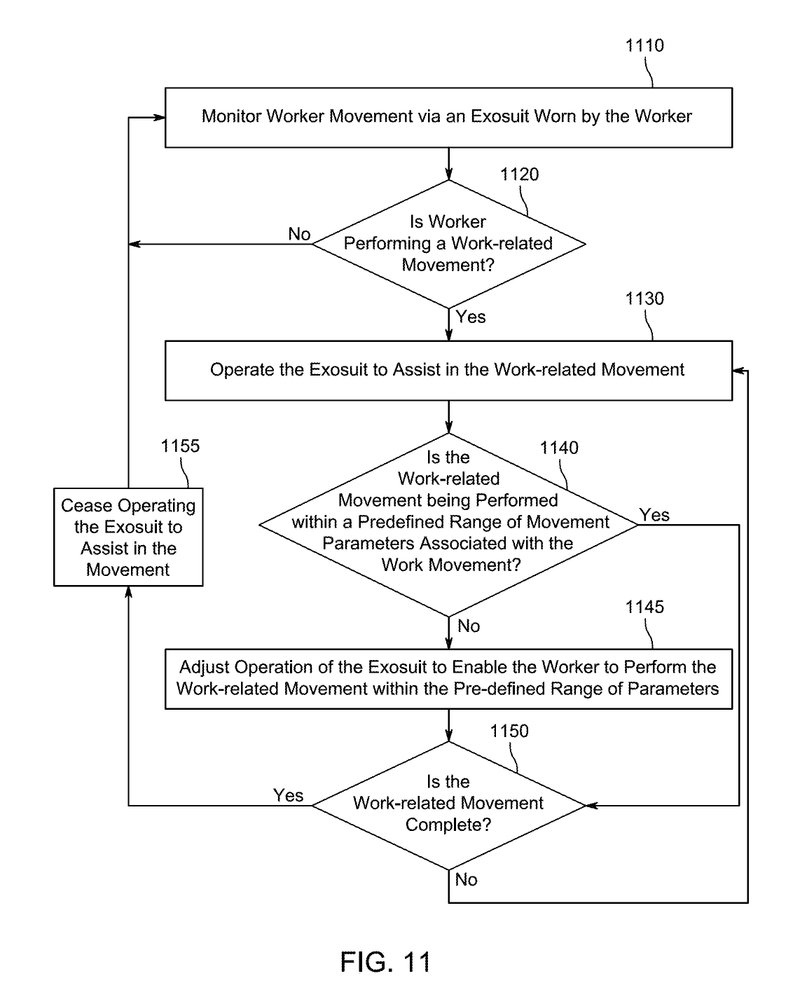

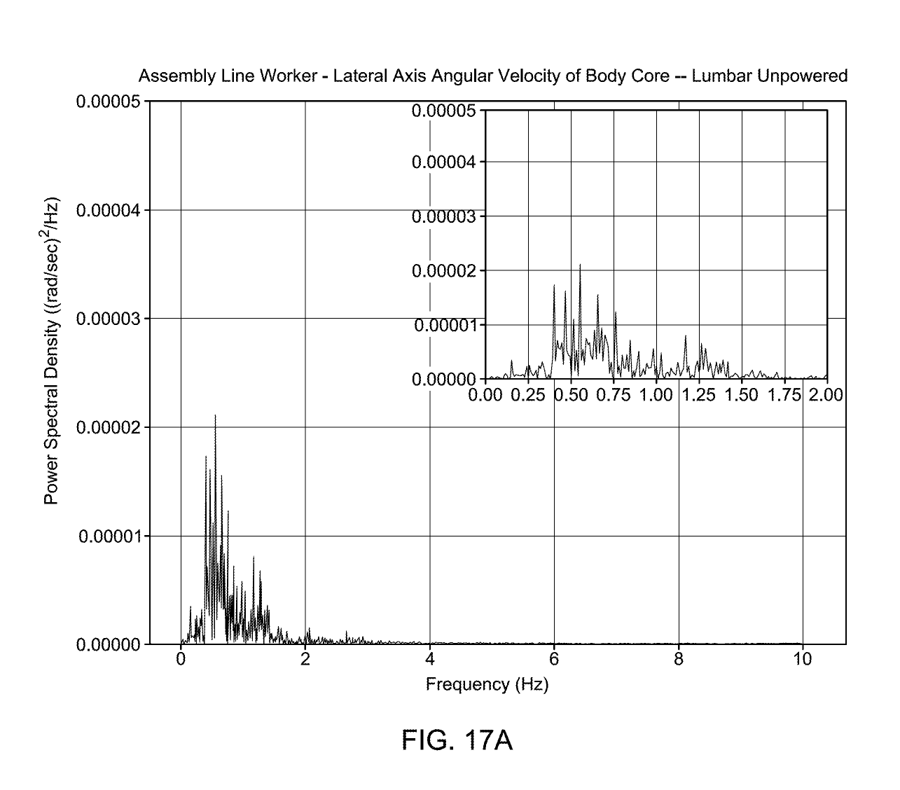

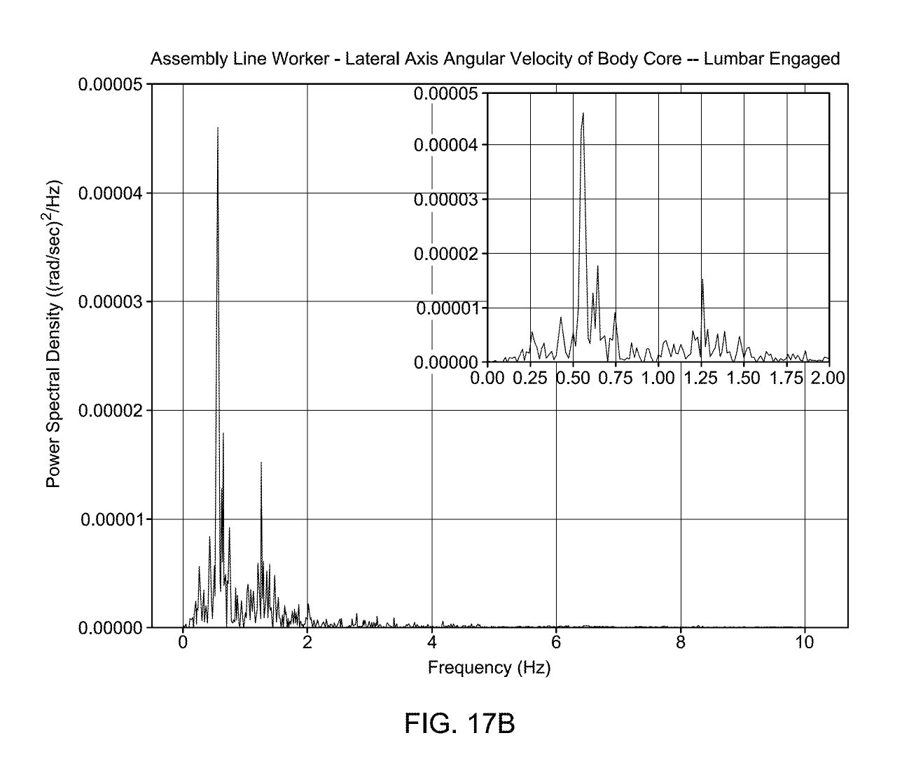

FIG. 11 shows illustrative worker movement monitoring process 1100 according to an embodiment. Process 1100 may be implemented by work activity module 570 of FIG. 5 and further implemented while a worker is wearing an exosuit. Process 1100 may begin at step 1110 by monitoring worker movement via an exosuit worn by the worker. At step 1120, a determination is made as to whether the worker is performing a work-related movement. For example, the work-related movement may be a lifting movement (e.g., lifting a pallet), a stocking movement (e.g., stocking shelves with goods), a laundry cleaning movement (e.g., ironing or folding), or any other movement. See, for example, the discussion below corresponding to FIGS. 17A and 17B for a use case in which exosuit lumbar support enables the worker to execute repetitive moves more efficiently. If the determination at step 1120 is NO, process 1100 may revert back to step 1110. If the determination is YES, process 1100 may operate the exosuit to assist in the work-related movement (as step 1130).

At step 1140, a determination is made as to whether the work-related movement is being performed with a predefined range of movement parameters associated with the work movement. The determination at step 1140 may resemble determination steps performed in training process 700 of FIG. 7. If the determination at step 1140 is NO, process 1100 may adjust operation of the exosuit to enable the worker to perform the work-related movement within the pre-defined range of parameters. At step 1150, process 1100 may determine whether the work-related movement is complete. If the determination at step 1150 is YES, process 1100 may cease assisting in the work-related movement at step 1155 and proceed back to step 1110. If the determination at step 1150 is NO, process 1130 may continue to operate the exosuit to assist in the work-related movement (step 1130). If the determination at step 1140 is YES, process 1100 may proceed to step 1150, as previously described.

It should be appreciated that the steps shown in FIG. 11 are merely illustrative and that additional steps may be added and the order of the steps may be rearranged.

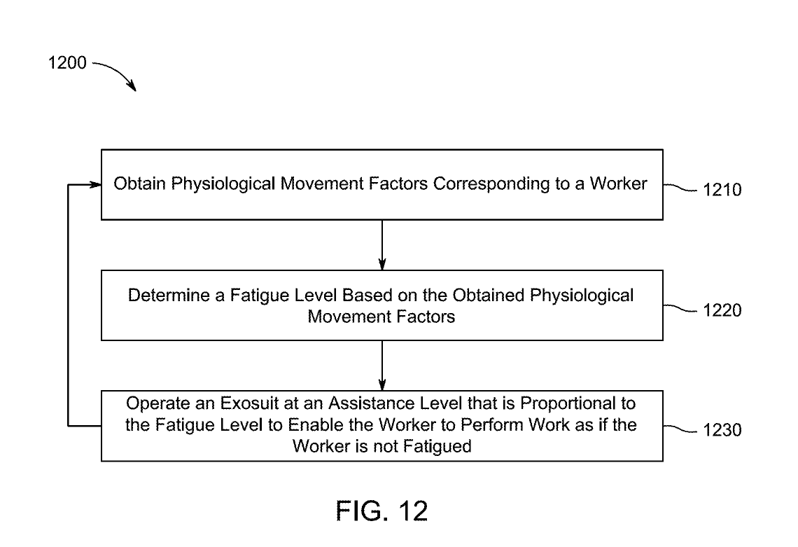

FIG. 12 shows illustrative maximizing worker productivity process 1200 according to an embodiment. Process 1200 may start at step 1210 by obtaining physiological movement factors corresponding to a worker using an exosuit. At step 1220, a fatigue level can be determined based on the obtained physiological movement factors. The fatigue level can be one of several fatigue levels that can be assigned to the worker. The fatigue levels can be linear or non-linear in progression. At step 1230, the exosuit can be operated at an assistance level that is proportional to the determined fatigue level to enable, for example, the worker to perform work as if the worker is not fatigued. Process 1200 may loop back to step 1210. Process 1200 can be advantageous as it allows the user to maintain his optimal productivity throughout his shift by dynamically assisting the worker based on his level of fatigue. The dynamic aspect of exosuit assistance can be particularly useful because the fatigue levels of the worker may change throughout the day. For example, at the beginning of the worker's shift, there may no signs of fatigue, but at hour 3 into the shift, fatigue may set in, but after a lunch break, there may be not fatigue initially. However, as the afternoon shift wears on, the worker's rate of fatigue may be faster than it was in the morning. The exosuit can detect the fatigue levels and accommodate accordingly. For example, in some embodiments, fatigue may be handled with exosuit feedback to inform the user that he or she is not maintaining proper posture. As a specific example, the exosuit may buzz the user to indicate that posture is incorrect.

It should be appreciated that the steps shown in FIG. 12 are merely illustrative and that additional steps may be added and the order of the steps may be rearranged.

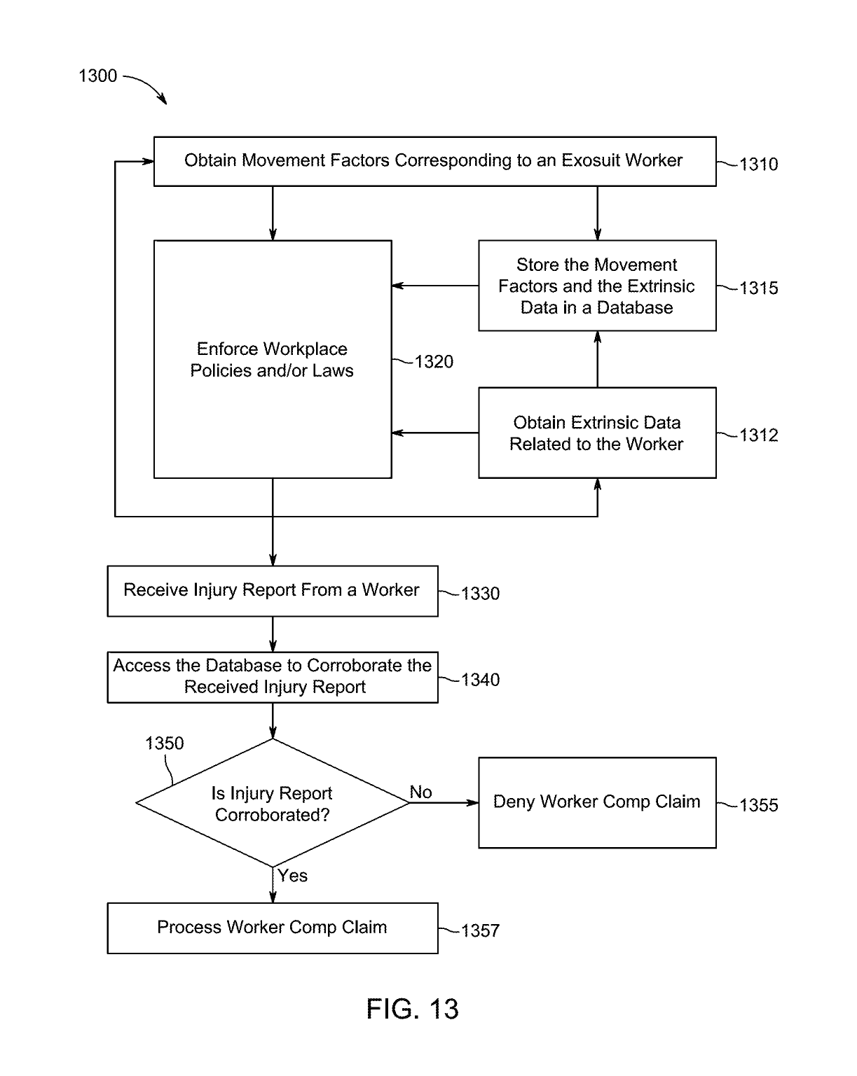

FIG. 13 shows illustrative policy enforcement process 1300 according to an embodiment. Process 1300 may be implemented by policy and law enforcement module 580 of FIG. 5. Process 1300 may start at step 1310 by obtaining movement factors corresponding to an exosuit worker. The movement factors can be stored in a database (step 1315). Extrinsic data related to the worker may be obtained at step 1312 and stored in the database at step 1315. The extrinsic data can include, for example, location data. The movement factors, extrinsic data, and stored data can be used to enforce workplace policies and/or laws (step 1320), via administrative or biomechanical measures. As an administrative policy, for example, if the worker has not taken his or her mandatory break, the exosuit may inform the worker to take a break. As a biomechanical policy, for example, the exosuit can independently enforce health and safety requirements regarding the manual handling of loads. The exosuit can help implement policies designed to reduce the risk of back injury to workers via a variety of metrics, including but not limited to measuring the physical effort required to lift loads (for both individual as well as the accumulation of lifting events), the frequency of lifting events, and the distribution of forces across various muscle groups. As another example, the exosuit can process the data to determine whether the worker is abiding by workplace policies and laws. Following step 1320, process 1300 may loop back to steps 1312 and 1310.

In some embodiments, process 1300 may receive an injury report from a worker at step 1330. The database may be accessed (at step 1340) to determine whether the injury report can be corroborated with the data stored in the database. For example, the injury report may allege that the worker slipped and fell on the factory floor, but the database may show that the worker was hanging out in the break room at the same time and that there is no presence of movement data suggesting that a fall took place. In another example, the exosuit can detect the onset of acute, chronic, and/or repetitive strain injuries by comparing biomechanical metrics over time. Acute injuries can be detected by observing a sharp transition in the quality of movement, while chronic and repetitive strain injuries have a gradual deterioration of movement. Furthermore, the exosuit can be used to independently verify the validity of the injury, by measuring the consistency of the worker's movement. Inconsistent and frequent transitions between healthy and unhealthy evaluations from the exosuit after the reported injury event can be used to identify false or fraudulent injury reports. If the injury report is corroborated at step 1350, a workers compensation claim can be processed at step 1367. If the injury report is not corroborated at step 1350, the claim can be denied at step 1355 or further investigation may be required.

It should be appreciated that the steps shown in FIG. 13 are merely illustrative and that additional steps may be added and the order of the steps may be rearranged.

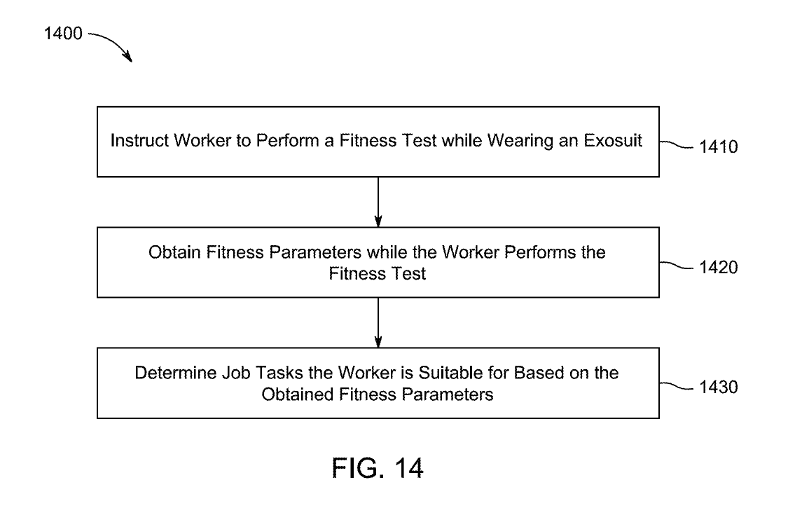

FIG. 14 shows illustrative job fitness determination process 1400 according to an embodiment. Process 1400 may start at step 1410 by instructing a worker to perform a fitness test while wearing an exosuit. The fitness test may include a series of movements that may be used on the job. Fitness parameters can be obtained while the worker performs the fitness test (step 1420). The fitness parameters can include biomechanical movement factors, for example. At step 1430, process 1400 can determine job tasks the worker is suitable for based on the obtained fitness parameters. For example, some people may be inherently predisposed to performed certain job tasks better than others. Some workers can be better suited for tasks that require standing for long periods of time, while other workers can be better suited for lifting heavy loads. The exosuit can be used to predict a worker's ability to succeed at a task by measuring their performance in a series of tests. A worker's propensity to stand for long periods of time can be measured via metrics which include, but are not limited to, analyzing their center of pressure via ground reaction force prediction, weight distribution balance, and frequency analysis of the oscillations in postural control; while a worker's propensity to lift heavy loads can be measured via metrics which include, but are not limited to, the dynamics of the lifting biomechanical form, the distribution of forces across various muscle groups, and the natural height of the lifting motion. Process 1400 can be used to assign workers to tasks that are better suited to them so that maximum productivity of each worker can be obtained.

In some embodiments, process 1420 may disable the PLSs in order to measure the worker's natural biomechanical motions. These measurements can provide a snapshot of the worker's natural abilities without the support of the exosuit. In some embodiments, process 1420 may actively enable the PLSs to evaluate the worker's biomechanical motions in an aided environment. These assisted measurements can be used to evaluate how much support is required from the exosuit by the worker and whether the assistance helps the long term aid from the exosuit adds productivity and ensures a healthy environment for the worker.

In some embodiments, process 1430 may be used to evaluate the respective roles for entire teams of workers so that their roles are optimized for their body types and biomechanical propensities. For example, securing a support bar may require two workers; one worker to hold the support bar, and another worker to fasten it in place. The fitness parameters measured in process 1420 can be used to determine that the worker with better standing form should hold the support bar, while the worker with better motor control should fasten the support bar. As fatigue is an important issue and workers' abilities may vary with respect to time due to fatigue, the exosuit can be used to provide a real-time, dynamic allocation of resources that optimizes the productivity of the team while also ensuring a safe environment.

It should be appreciated that the steps shown in FIG. 14 are merely illustrative and that additional steps may be added and the order of the steps may be rearranged.

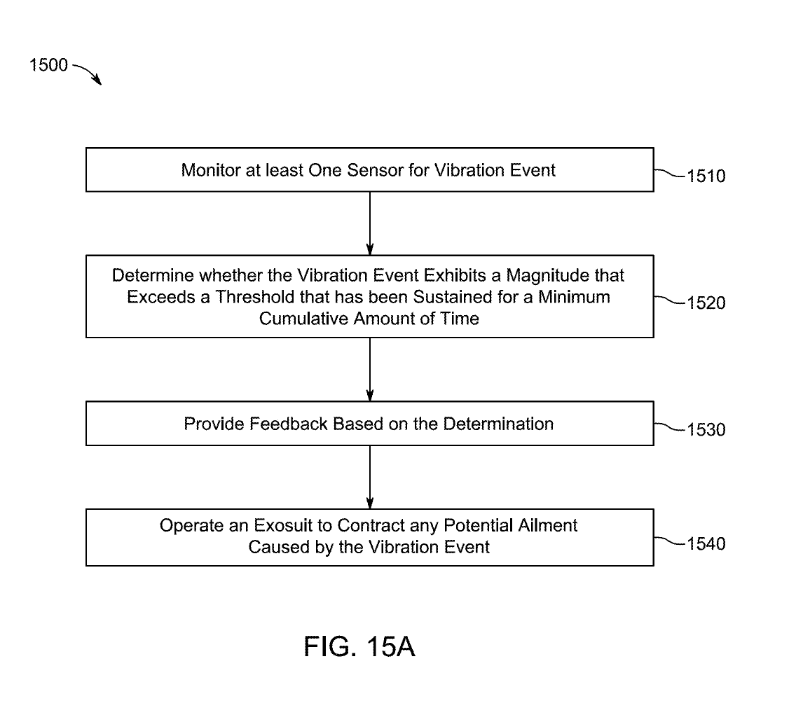

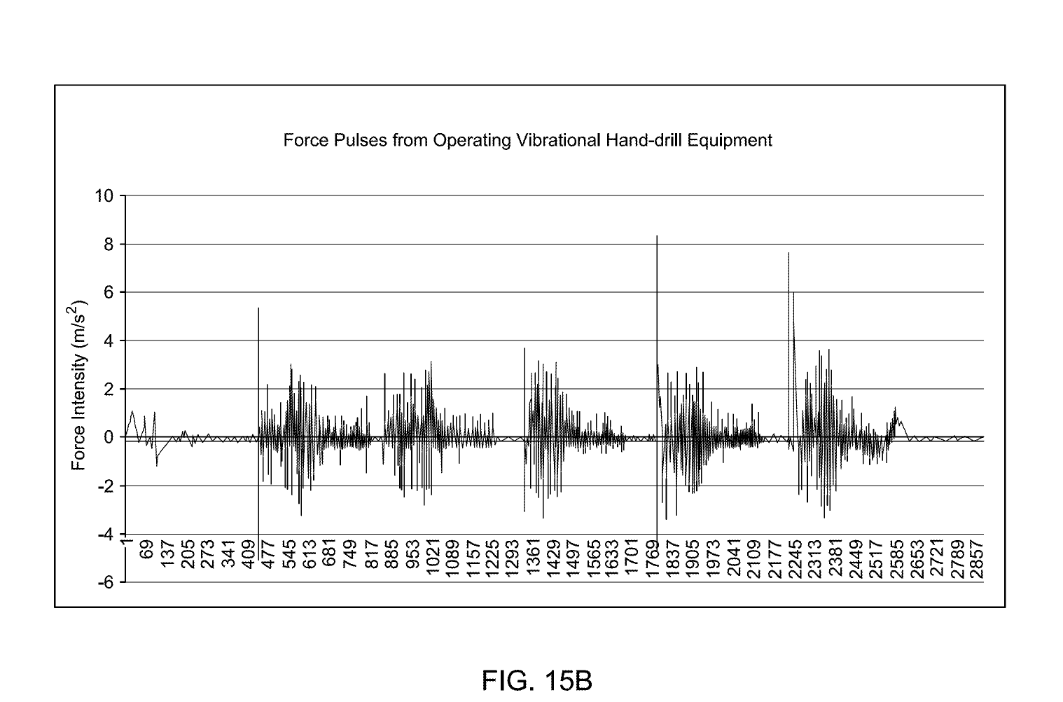

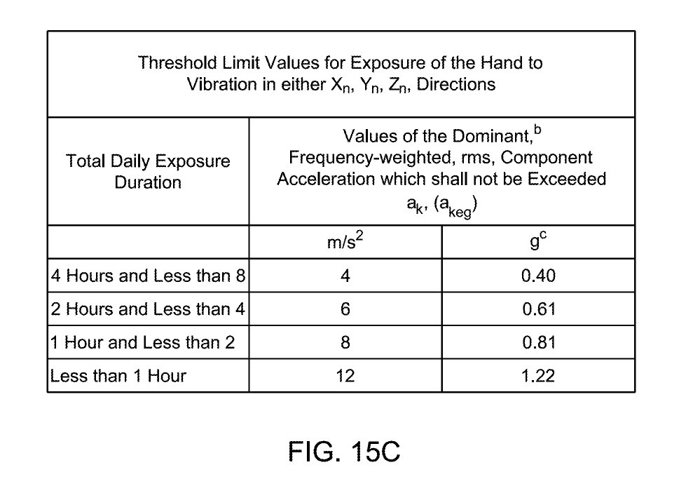

FIG. 15A shows illustrative vibration monitoring process 1500 according to an embodiment. Process 1500 may monitor at least one sensor for a vibration event. For example, process 1500 may perform high frequency vibration analysis and the biomechanical analysis based on date obtained from the sensors. If the user is using a power drill, for example, the force intensities being translated into the arms and body of the user using the drill may resemble the force profile illustrated in FIG. 15B. The raw accelerations and magnitude vector of the accelerations, such as those shown in FIG. 15B can serve as the raw data for vibration analysis. Vibration analysis may be performed, for example, by running a discrete Fourier transform (DFT) to convert the time series data to the frequency domain for further analysis. The analysis can determine whether the vibration event exhibits a magnitude that exceeds a threshold that has been sustained for a minimum cumulative amount of time. For example, illustrative magnitude and time thresholds are expressed in FIG. 15C. In particular, thresholds limits in FIG. 15C are recommended by the American Conference of Governmental Industrial Hygienists on exposure duration to various vibration intensities.

At step 1530, feedback can be provided to the user based on the determination (obtained in step 1520). The feedback can include a notification for the operator to take a break or find another person to replace him. In some embodiments, the feedback may shut the equipment down to prevent injury. In some embodiments, the feedback may be provided via headphones that the worker uses to drown out the noise while operating heavy machinery. In other embodiments, feedback can include notice of the proper biomechanics for operating equipment to ensure worker safety.

At step 1540, an exosuit can operated to counteract any potential ailments that can be caused by the vibration event. For example, the exosuit can ensure that the user is maintaining proper posture while using the equipment, or that the user is holding or using the equipment correctly. In some embodiments, the exosuit can apply vibration haptics to counteract the vibration being induced by the equipment.

It should be appreciated that the steps shown in FIG. 15 are merely illustrative and that additional steps may be added and the order of the steps may be rearranged.

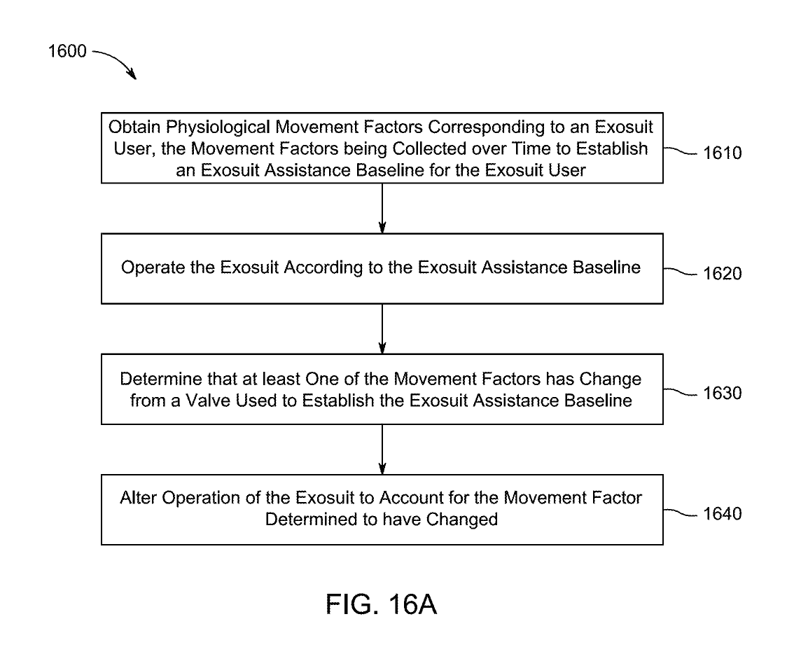

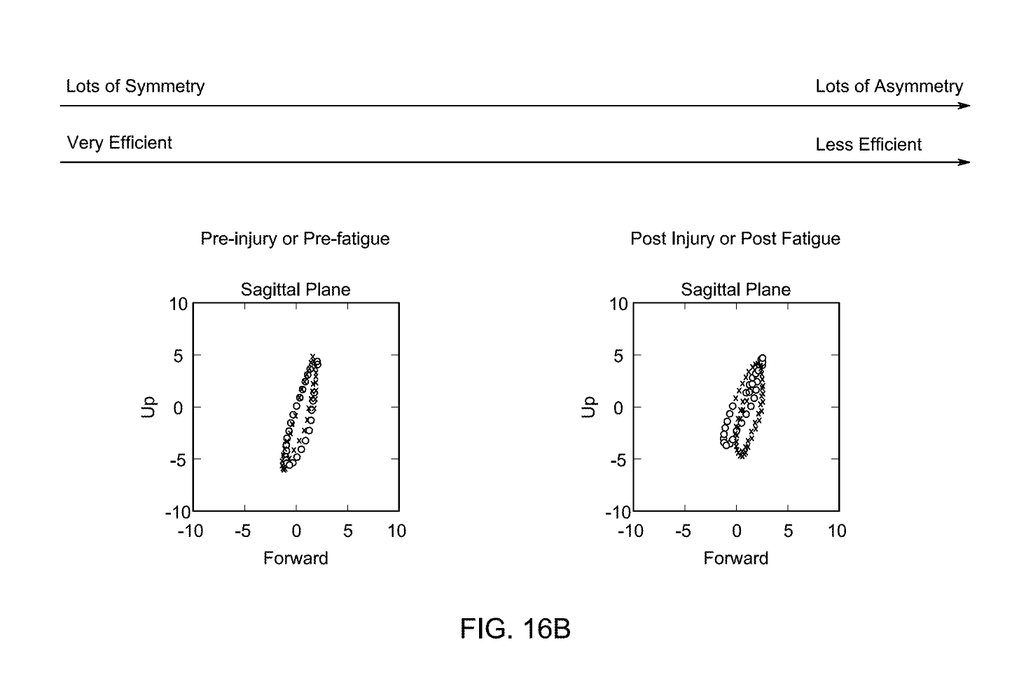

FIG. 16A shows illustrative process 1600 for using an exosuit to assist a user according to an embodiment. Process 1600 may be implemented by a control module according to an embodiment discussed herein. Process 1600 may start at step 1610 by obtaining physiological or bio-mechanical movement factors corresponding to an exosuit user. The movement factors can be collected over time to establish an exosuit assistance baseline for the exosuit user. The exosuit can be operated according to the exosuit assistance baseline at step 1620. At step 1630, a determination can be made that at least one of the movement factors changed from a value used to establish the exosuit assistance baseline. For example, the user is fatigued or experienced an injury, resulting in different movement factors. For example, FIG. 16B shows movement factor data corresponding to a person before injury (or fatigue) and after injury (or fatigue). The data represents movement data of each foot during a stride. The circles in FIG. 16B can correspond to one foot and the x's can correspond to other foot. The before event data shows that both feet have nearly symmetrical data, and the after event data shows that feet movements are more asymmetric. The more symmetric the movement is, the more efficient the movement. The before event data may be used as one of several factors to establish the exosuit assistance baseline. When the movement data shows that one of the factors has changed, as shown by the after event data, process 1640 can alter the operation of the exosuit to account for the movement factor determined to have changed, as indicated by step 1640.

It should be appreciated that the steps shown in FIG. 16 is merely illustrative and that additional steps may be added and the order of the steps may be rearranged.

FIGS. 17A and 17B show illustrative “before” and “after” plots of movement data for an assembly-line worker according to an embodiment. In this specific example, the data shows an effect of engaging lumbar support, via an exosuit, of an assembly-line worker who is folding towels and placing the folded units on a conveyor belt. The plots show the effect on the worker's forward pitch angular velocity, that is, on the speed at which the worker leans forward and then straightens up as she picks up, folds, and sets down the towels. The plots show the frequency spectrum of the motion. The before plot (“lumbar unpowered”, FIG. 17A) shows a wide range of frequencies contributing to the overall motion, while the after plot (“lumbar engaged”, FIG. 17B) shows that one frequency (near 0.55 Hz) becomes dominant as others are reduced in amplitude. This is presumably an effect of the lumbar support, encouraging the worker to bend forward/back less from activating her lower back muscles and more by activating her hip muscles. One might reasonably expect that reducing long-term, repetitive strain on the lower back (by encouraging the hips to provide more of the rotation) would be helpful in reducing lower-back injuries.

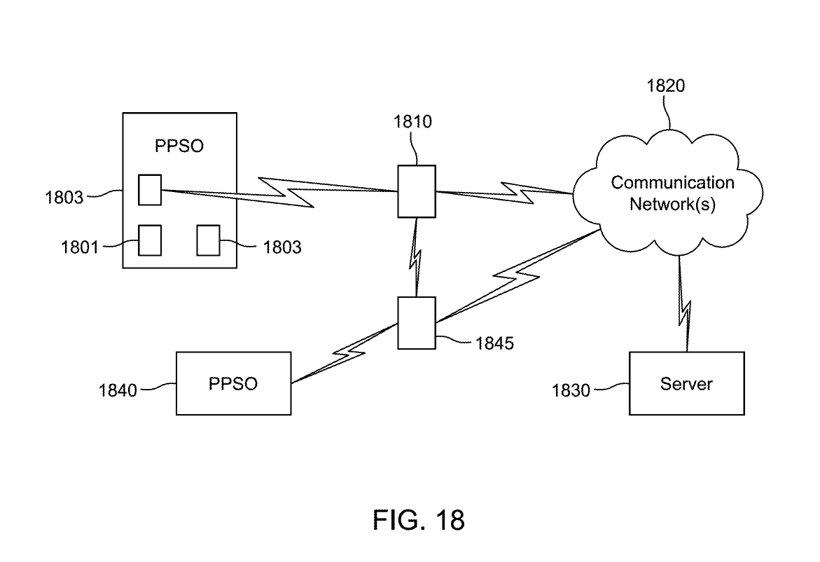

FIG. 18 illustrates an example exosuit 1800 that includes actuators 1801, sensors 1803, and a controller configured to operate elements of exosuit 1800 (e.g., 1801, 1803) to enable functions of the exosuit 1800. The controller 1805 is configured to communicate wirelessly with a user interface 1810. The user interface 1810 is configured to present information to a user (e.g., a wearer of the exosuit 1800) and to the controller 1805 of the flexible exosuit or to other systems. The user interface 1810 can be involved in controlling and/or accessing information from elements of the exosuit 1800. For example, an application being executed by the user interface 1810 can access data from the sensors 1803, calculate an operation (e.g., to apply dorsiflexion stretch) of the actuators 1801, and transmit the calculated operation to the exosuit 1800. The user interface 1810 can additionally be configured to enable other functions; for example, the user interface 1810 can be configured to be used as a cellular telephone, a portable computer, an entertainment device, or to operate according to other applications.

The user interface 1810 can be configured to be removably mounted to the exosuit 1800 (e.g., by straps, magnets, Velcro, charging and/or data cables). Alternatively, the user interface 1810 can be configured as a part of the exosuit 1800 and not to be removed during normal operation. In some examples, a user interface can be incorporated as part of the exosuit 1800 (e.g., a touchscreen integrated into a sleeve of the exosuit 1800) and can be used to control and/or access information about the exosuit 1800 in addition to using the user interface 1810 to control and/or access information about the exosuit 1800. In some examples, the controller 1805 or other elements of the exosuit 1800 are configured to enable wireless or wired communication according to a standard protocol (e.g., Bluetooth, ZigBee, WiFi, LTE or other cellular standards, IRdA, Ethernet) such that a variety of systems and devices can be made to operate as the user interface 1810 when configured with complementary communications elements and computer-readable programs to enable such functionality.

The exosuit 1800 can be configured as described in example embodiments herein or in other ways according to an application. The exosuit 1800 can be operated to enable a variety of applications. The exosuit 1800 can be operated to enhance the strength of a wearer by detecting motions of the wearer (e.g., using sensors 1803) and responsively applying torques and/or forces to the body of the wearer (e.g., using actuators 1801) to increase the forces the wearer is able to apply to his/her body and/or environment. The exosuit 1800 can be operated to train a wearer to perform certain physical activities. For example, the exosuit 1800 can be operated to enable rehabilitative therapy of a wearer. The exosuit 1800 can operate to amplify motions and/or forces produced by a wearer undergoing therapy in order to enable the wearer to successfully complete a program of rehabilitative therapy. Additionally or alternatively, the exosuit 1800 can be operated to prohibit disordered movements of the wearer and/or to use the actuators 1801 and/or other elements (e.g., haptic feedback elements) to indicate to the wearer a motion or action to perform and/or motions or actions that should not be performed or that should be terminated. Similarly, other programs of physical training (e.g., dancing, skating, other athletic activities, vocational training) can be enabled by operation of the exosuit 1800 to detect motions, torques, or forces generated by a wearer and/or to apply forces, torques, or other haptic feedback to the wearer. Other applications of the exosuit 1800 and/or user interface 1810 are anticipated.

The user interface 1810 can additionally communicate with communications network(s) 1820. For example, the user interface 1810 can include a WiFi radio, an LTE transceiver or other cellular communications equipment, a wired modem, or some other elements to enable the user interface 1810 and exosuit 1800 to communicate with the Internet. The user interface 1810 can communicate through the communications network 1820 with a server 1830. Communication with the server 1830 can enable functions of the user interface 1810 and exosuit 1800. In some examples, the user interface 1810 can upload telemetry data (e.g., location, configuration of elements 1801, 1803 of the exosuit 1800, physiological data about a wearer of the exosuit 1800) to the server 1830.

In some examples, the server 1830 can be configured to control and/or access information from elements of the exosuit 1800 (e.g., 1801, 1803) to enable some application of the exosuit 1800. For example, the server 1830 can operate elements of the exosuit 1800 to move a wearer out of a dangerous situation if the wearer was injured, unconscious, or otherwise unable to move themselves and/or operate the exosuit 1800 and user interface 1810 to move themselves out of the dangerous situation. Other applications of a server in communications with a exosuit are anticipated.

The user interface 1810 can be configured to communicate with a second user interface 1845 in communication with and configured to operate a second flexible exosuit 1840. Such communication can be direct (e.g., using radio transceivers or other elements to transmit and receive information over a direct wireless or wired link between the user interface 1810 and the second user interface 1845). Additionally or alternatively, communication between the user interface 1810 and the second user interface 1845 can be facilitated by communications network(s) 1820 and/or a server 1830 configured to communicate with the user interface 1810 and the second user interface 1845 through the communications network(s) 1820.

Communication between the user interface 1810 and the second user interface 1845 can enable applications of the exosuit 1800 and second exosuit 1840. In some examples, actions of the exosuit 1800 and second flexible exosuit 1840 and/or of wearers of the exosuit 1800 and second exosuit 1840 can be coordinated. For example, the exosuit 1800 and second exosuit 1840 can be operated to coordinate the lifting of a heavy object by the wearers. The timing of the lift, and the degree of support provided by each of the wearers and/or the exosuit 1800 and second exosuit 1840 can be controlled to increase the stability with which the heavy object was carried, to reduce the risk of injury of the wearers, or according to some other consideration. Coordination of actions of the exosuit 1800 and second exosuit 1840 and/or of wearers thereof can include applying coordinated (in time, amplitude, or other properties) forces and/or torques to the wearers and/or elements of the environment of the wearers and/or applying haptic feedback (though actuators of the exosuits 1800, 1840, through dedicated haptic feedback elements, or through other methods) to the wearers to guide the wearers toward acting in a coordinated manner.

Coordinated operation of the exosuit 1800 and second exosuit 1840 can be implemented in a variety of ways. In some examples, one exosuit (and the wearer thereof) can act as a master, providing commands or other information to the other exosuit such that operations of the exosuit 1800, 1840 are coordinated. For example, the exosuit 1800, 1840 can be operated to enable the wearers to dance (or to engage in some other athletic activity) in a coordinated manner. One of the exosuits can act as the ‘lead’, transmitting timing or other information about the actions performed by the ‘lead’ wearer to the other exosuit, enabling coordinated dancing motions to be executed by the other wearer. In some examples, a first wearer of a first exosuit can act as a trainer, modeling motions or other physical activities that a second wearer of a second exosuit can learn to perform. The first exosuit can detect motions, torques, forces, or other physical activities executed by the first wearer and can send information related to the detected activities to the second exosuit. The second exosuit can then apply forces, torques, haptic feedback, or other information to the body of the second wearer to enable the second wearer to learn the motions or other physical activities modeled by the first wearer. In some examples, the server 1830 can send commands or other information to the exosuits 1800, 1840 to enable coordinated operation of the exosuits 1800, 1840.

The exosuit 1800 can be operated to transmit and/or record information about the actions of a wearer, the environment of the wearer, or other information about a wearer of the exosuit 1800. In some examples, kinematics related to motions and actions of the wearer can be recorded and/or sent to the server 1830. These data can be collected for medical, scientific, entertainment, social media, or other applications. The data can be used to operate a system. For example, the exosuit 1800 can be configured to transmit motions, forces, and/or torques generated by a user to a robotic system (e.g., a robotic arm, leg, torso, humanoid body, or some other robotic system) and the robotic system can be configured to mimic the activity of the wearer and/or to map the activity of the wearer into motions, forces, or torques of elements of the robotic system. In another example, the data can be used to operate a virtual avatar of the wearer, such that the motions of the avatar mirrored or were somehow related to the motions of the wearer. The virtual avatar can be instantiated in a virtual environment, presented to an individual or system with which the wearer is communicating, or configured and operated according to some other application.

Conversely, the exosuit 1800 can be operated to present haptic or other data to the wearer. In some examples, the actuators 1801 (e.g., twisted string actuators, exotendons) and/or haptic feedback elements (e.g., EPAM haptic elements) can be operated to apply and/or modulate forces applied to the body of the wearer to indicate mechanical or other information to the wearer. For example, the activation in a certain pattern of a haptic element of the exosuit 1800 disposed in a certain location of the exosuit 1800 can indicate that the wearer had received a call, email, or other communications. In another example, a robotic system can be operated using motions, forces, and/or torques generated by the wearer and transmitted to the robotic system by the exosuit 1800. Forces, moments, and other aspects of the environment and operation of the robotic system can be transmitted to the exosuit 1800 and presented (using actuators 1801 or other haptic feedback elements) to the wearer to enable the wearer to experience force-feedback or other haptic sensations related to the wearer's operation of the robotic system. In another example, haptic data presented to a wearer can be generated by a virtual environment, e.g., an environment containing an avatar of the wearer that is being operated based on motions or other data related to the wearer that is being detected by the exosuit 1800.

Note that the exosuit 1800 illustrated in FIG. 18 is only one example of a exosuit that can be operated by control electronics, software, or algorithms described herein. Control electronics, software, or algorithms as described herein can be configured to control flexible exosuits or other mechatronic and/or robotic system having more, fewer, or different actuators, sensors or other elements. Further, control electronics, software, or algorithms as described herein can be configured to control exosuits configured similarly to or differently from the illustrated exosuit 1800. Further, control electronics, software, or algorithms as described herein can be configured to control flexible exosuits having reconfigurable hardware (i.e., exosuits that are able to have actuators, sensors, or other elements added or removed) and/or to detect a current hardware configuration of the flexible exosuits using a variety of methods.

A controller of a exosuit and/or computer-readable programs executed by the controller can be configured to provide encapsulation of functions and/or components of the flexible exosuit. That is, some elements of the controller (e.g., subroutines, drivers, services, daemons, functions) can be configured to operate specific elements of the exosuit (e.g., a twisted string actuator, a haptic feedback element) and to allow other elements of the controller (e.g., other programs) to operate the specific elements and/or to provide abstracted access to the specific elements (e.g., to translate a command to orient an actuator in a commanded direction into a set of commands sufficient to orient the actuator in the commanded direction). This encapsulation can allow a variety of services, drivers, daemons, or other computer-readable programs to be developed for a variety of applications of a flexible exosuits. Further, by providing encapsulation of functions of a flexible exosuit in a generic, accessible manner (e.g., by specifying and implementing an application programming interface (API) or other interface standard), computer-readable programs can be created to interface with the generic, encapsulated functions such that the computer-readable programs can enable operating modes or functions for a variety of differently-configured exosuit, rather than for a single type or model of flexible exosuit. For example, a virtual avatar communications program can access information about the posture of a wearer of a flexible exosuit by accessing a standard exosuit API. Differently-configured exosuits can include different sensors, actuators, and other elements, but can provide posture information in the same format according to the API. Other functions and features of a flexible exosuit, or other robotic, exoskeletal, assistive, haptic, or other mechatronic system, can be encapsulated by APIs or according to some other standardized computer access and control interface scheme.

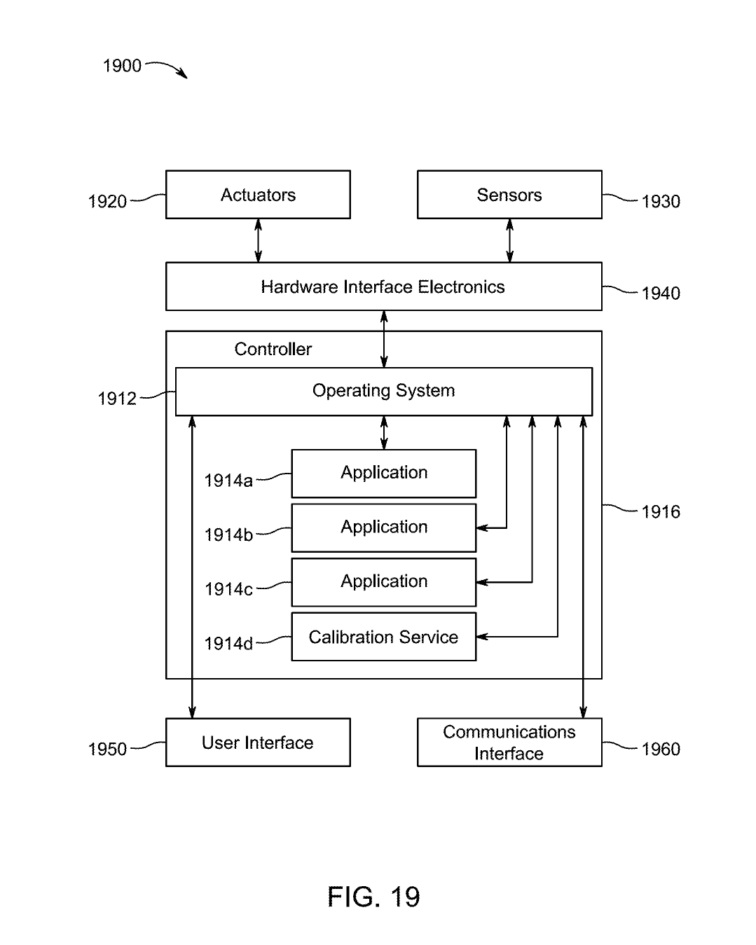

FIG. 19 is a schematic illustrating elements of a exosuit 1900 and a hierarchy of control or operating the exosuit 1900. The flexible exosuit includes actuators 1920 and sensors 1930 configured to apply forces and/or torques to and detect one or more properties of, respectively, the exosuit 1900, a wearer of the exosuit 1900, and/or the environment of the wearer. The exosuit 1900 additionally includes a controller 1910 configured to operate the actuators 1920 and sensors 1930 by using hardware interface electronics 1940. The hardware electronics interface 1940 includes electronics configured to interface signals from and to the controller 1910 with signals used to operate the actuators 1920 and sensors 1930. For example, the actuators 1920 can include exotendons, and the hardware interface electronics 1940 can include high-voltage generators, high-voltage switches, and high-voltage capacitance meters to clutch and un-clutch the exotendons and to report the length of the exotendons. The hardware interface electronics 1940 can include voltage regulators, high voltage generators, amplifiers, current detectors, encoders, magnetometers, switches, controlled-current sources, DACs, ADCs, feedback controllers, brushless motor controllers, or other electronic and mechatronic elements.

The controller 1910 additionally operates a user interface 1950 that is configured to present information to a user and/or wearer of the exosuit 1900 and a communications interface 1960 that is configured to facilitate the transfer of information between the controller 1910 and some other system (e.g., by transmitting a wireless signal). Additionally or alternatively, the user interface 1950 can be part of a separate system that is configured to transmit and receive user interface information to/from the controller 1910 using the communications interface 1960 (e.g., the user interface 1950 can be part of a cellphone).

The controller 1910 is configured to execute computer-readable programs describing functions of the flexible exosuit 1912. Among the computer-readable programs executed by the controller 1910 are an operating system 1912, applications 1914 a, 1914 b, 1914 c, and a calibration service 1916. The operating system 1912 manages hardware resources of the controller 1910 (e.g., I/O ports, registers, timers, interrupts, peripherals, memory management units, serial and/or parallel communications units) and, by extension, manages the hardware resources of the exosuit 1900. The operating system 1912 is the only computer-readable program executed by the controller 1910 that has direct access to the hardware interface electronics 1940 and, by extension, the actuators 1920 and sensors 1930 of the exosuit 1900.

The applications 1914 a, 1914 b, 1914 are computer-readable programs that describe some function, functions, operating mode, or operating modes of the exosuit 1900. For example, application 1914 a can describe a process for transmitting information about the wearer's posture to update a virtual avatar of the wearer that includes accessing information on a wearer's posture from the operating system 1912, maintaining communications with a remote system using the communications interface 1960, formatting the posture information, and sending the posture information to the remote system. The calibration service 1916 is a computer-readable program describing processes to store parameters describing properties of wearers, actuators 1920, and/or sensors 1930 of the exosuit 1900, to update those parameters based on operation of the actuators 1920, and/or sensors 1930 when a wearer is using the exosuit 1900, to make the parameters available to the operating system 1912 and/or applications 1914 a, 1914 b, 1914 c, and other functions relating to the parameters. Note that applications 1914 a, 1914 b, 1914 and calibration service 1916 are intended as examples of computer-readable programs that can be run by the operating system 1912 of the controller 1910 to enable functions or operating modes of a exosuit 1900.

The operating system 1912 can provide for low-level control and maintenance of the hardware (e.g., 1920, 1930, 1940). In some examples, the operating system 1912 and/or hardware interface electronics 1540 can detect information about the exosuit 1900, the wearer, and/or the wearer's environment from one or more sensors 1930 at a constant specified rate. The operating system 1912 can generate an estimate of one or more states or properties of the exosuit 1900 or components thereof using the detected information. The operating system 1912 can update the generated estimate at the same rate as the constant specified rate or at a lower rate. The generated estimate can be generated from the detected information using a filter to remove noise, generate an estimate of an indirectly-detected property, or according to some other application. For example, the operating system 1912 can generate the estimate from the detected information using a Kalman filter to remove noise and to generate an estimate of a single directly or indirectly measured property of the exosuit 1900, the wearer, and/or the wearer's environment using more than one sensor. In some examples, the operating system can determine information about the wearer and/or exosuit 1900 based on detected information from multiple points in time. For example, the operating system 1900 can determine an eversion stretch and dorsiflexion stretch.

In some examples, the operating system 1912 and/or hardware interface electronics 1940 can operate and/or provide services related to operation of the actuators 1920. That is, in case where operation of the actuators 1920 requires the generation of control signals over a period of time, knowledge about a state or states of the actuators 1920, or other considerations, the operating system 1912 and/or hardware interface electronics 1940 can translate simple commands to operate the actuators 1920 (e.g., a command to generate a specified level of force using a twisted string actuator (TSA) of the actuators 1920) into the complex and/or state-based commands to the hardware interface electronics 1940 and/or actuators 1920 necessary to effect the simple command (e.g., a sequence of currents applied to windings of a motor of a TSA, based on a starting position of a rotor determined and stored by the operating system 1910, a relative position of the motor detected using an encoder, and a force generated by the TSA detected using a load cell).

In some examples, the operating system 1912 can further encapsulate the operation of the exosuit 1900 by translating a system-level simple command (e.g., a commanded level of force tension applied to the footplate) into commands for multiple actuators, according to the configuration of the exosuit 1900. This encapsulation can enable the creation of general-purpose applications that can effect a function of an exosuit (e.g., allowing a wearer of the exosuit to stretch his foot) without being configured to operate a specific model or type of exosuit (e.g., by being configured to generate a simple force production profile that the operating system 1912 and hardware interface electronics 1940 can translate into actuator commands sufficient to cause the actuators 1920 to apply the commanded force production profile to the footplate).

The operating system 1912 can act as a standard, multi-purpose platform to enable the use of a variety of exosuits having a variety of different hardware configurations to enable a variety of mechatronic, biomedical, human interface, training, rehabilitative, communications, and other applications. The operating system 1912 can make sensors 1930, actuators 1920, or other elements or functions of the exosuit 1900 available to remote systems in communication with the exosuit 1900 (e.g., using the communications interface 1960) and/or a variety of applications, daemons, services, or other computer-readable programs being executed by operating system 1912. The operating system 1912 can make the actuators, sensors, or other elements or functions available in a standard way (e.g., through an API, communications protocol, or other programmatic interface) such that applications, daemons, services, or other computer-readable programs can be created to be installed on, executed by, and operated to enable functions or operating modes of a variety of flexible exosuits having a variety of different configurations. The API, communications protocol, or other programmatic interface made available by the operating system 1912 can encapsulate, translate, or otherwise abstract the operation of the exosuit 1900 to enable the creation of such computer-readable programs that are able to operate to enable functions of a wide variety of differently-configured flexible exosuits.

Additionally or alternatively, the operating system 1912 can be configured to operate a modular flexible exosuit system (i.e., a flexible exosuit system wherein actuators, sensors, or other elements can be added or subtracted from a flexible exosuit to enable operating modes or functions of the flexible exosuit). In some examples, the operating system 1912 can determine the hardware configuration of the exosuit 1900 dynamically and can adjust the operation of the exosuit 1900 relative to the determined current hardware configuration of the exosuit 1900. This operation can be performed in a way that was ‘invisible’ to computer-readable programs (e.g., 1914 a, 1914 b, 1914 c) accessing the functionality of the exosuit 1900 through a standardized programmatic interface presented by the operating system 1912. For example, the computer-readable program can indicate to the operating system 1912, through the standardized programmatic interface, that a specified level of torque was to be applied to an ankle of a wearer of the exosuit 1900. The operating system 1912 can responsively determine a pattern of operation of the actuators 1920, based on the determined hardware configuration of the exosuit 1900, sufficient to apply the specified level of torque to the ankle of the wearer.

In some examples, the operating system 1912 and/or hardware interface electronics 1940 can operate the actuators 1920 to ensure that the exosuit 1900 does not operate to directly cause the wearer to be injured and/or elements of the exosuit 1900 to be damaged. In some examples, this can include not operating the actuators 1920 to apply forces and/or torques to the body of the wearer that exceeded some maximum threshold. This can be implemented as a watchdog process or some other computer-readable program that can be configured (when executed by the controller 1910) to monitor the forces being applied by the actuators 1920 (e.g., by monitoring commands sent to the actuators 1920 and/or monitoring measurements of forces or other properties detected using the sensors 1930) and to disable and/or change the operation of the actuators 1920 to prevent injury of the wearer. Additionally or alternatively, the hardware interface electronics 1940 can be configured to include circuitry to prevent excessive forces and/or torques from being applied to the wearer (e.g., by channeling to a comparator the output of a load cell that is configured to measure the force generated by a TSA, and configuring the comparator to cut the power to the motor of the TSA when the force exceeded a specified level).

In some examples, operating the actuators 1920 to ensure that the exosuit 1900 does not damage itself can include a watchdog process or circuitry configured to prevent over-current, over-load, over-rotation, or other conditions from occurring that can result in damage to elements of the exosuit 1900. For example, the hardware interface electronics 1940 can include a metal oxide varistor, breaker, shunt diode, or other element configured to limit the voltage and/or current applied to a winding of a motor.

Note that the above functions described as being enabled by the operating system 1912 can additionally or alternatively be implemented by applications 1914 a, 1914 b, 1914 c, services, drivers, daemons, or other computer-readable programs executed by the controller 1900. The applications, drivers, services, daemons, or other computer-readable programs can have special security privileges or other properties to facilitate their use to enable the above functions.

The operating system 1912 can encapsulate the functions of the hardware interface electronics 1940, actuators 1920, and sensors 1930 for use by other computer-readable programs (e.g., applications 1914 a, 1914 b, 1914 c, calibration service 1916), by the user (through the user interface 1950), and/or by some other system (i.e., a system configured to communicate with the controller 1910 through the communications interface 1960). The encapsulation of functions of the exosuit 1900 can take the form of application programming interfaces (APIs), i.e., sets of function calls and procedures that an application running on the controller 1910 can use to access the functionality of elements of the exosuit 1900. In some examples, the operating system 1912 can make available a standard ‘exosuit API’ to applications being executed by the controller 1910. The ‘exosuit API’ can enable applications 1914 a, 1914 b, 1914 c to access functions of the exosuit 1900 without requiring those applications 1914 a, 1914 b, 1914 c to be configured to generate whatever complex, time-dependent signals are necessary to operate elements of the exosuit 1900 (e.g., actuators 1920, sensors 1930).

The ‘exosuit API’ can allow applications 1914 a, 1914 b, 1914 c to send simple commands to the operating system 1912 (e.g., ‘begin storing mechanical energy from the ankle of the wearer when the foot of the wearer contacts the ground’) in such that the operating system 1912 can interpret those commands and generate the command signals to the hardware interface electronics 1940 or other elements of the exosuit 1900 that are sufficient to effect the simple commands generated by the applications 1914 a, 1914 b, 1914 c (e.g., determining whether the foot of the wearer has contacted the ground based on information detected by the sensors 1930, responsively applying high voltage to an exotendon that crosses the user's ankle).

The ‘exosuit API’ can be an industry standard (e.g., an ISO standard), a proprietary standard, an open-source standard, or otherwise made available to individuals that can then produce applications for exosuits. The ‘exosuit API’ can allow applications, drivers, services, daemons, or other computer-readable programs to be created that are able to operate a variety of different types and configurations of exosuits by being configured to interface with the standard ‘exosuit API’ that is implemented by the variety of different types and configurations of exosuits. Additionally or alternatively, the ‘exosuit API’ can provide a standard encapsulation of individual exosuit-specific actuators (i.e., actuators that apply forces to specific body segments, where differently-configured exosuits may not include an actuator that applies forces to the same specific body segments) and can provide a standard interface for accessing information on the configuration of whatever exosuit is providing the ‘exosuit API’. An application or other program that accesses the ‘exosuit API’ can access data about the configuration of the exosuit (e.g., locations and forces between body segments generated by actuators, specifications of actuators, locations and specifications of sensors) and can generate simple commands for individual actuators (e.g., generate a force of 30 newtons for 50 milliseconds) based on a model of the exosuit generated by the application and based on the information on the accessed data about the configuration of the exosuit. Additional or alternate functionality can be encapsulated by an ‘exosuit API’ according to an application.

Applications 1914 a, 1914 b, 1914 c can individually enable all or parts of the functions and operating modes of a flexible exosuit described herein. For example, an application can enable haptic control of a robotic system by transmitting postures, forces, torques, and other information about the activity of a wearer of the exosuit 1900 and by translating received forces and torques from the robotic system into haptic feedback applied to the wearer (i.e., forces and torques applied to the body of the wearer by actuators 1920 and/or haptic feedback elements). In another example, an application can enable a wearer to locomote more efficiently by submitting commands to and receiving data from the operating system 1912 (e.g., through an API) such that actuators 1920 of the exosuit 1900 assist the movement of the user, extract negative work from phases of the wearer's locomotion and inject the stored work to other phases of the wearer's locomotion, or other methods of operating the exosuit 1900. Applications can be installed on the controller 1910 and/or on a computer-readable storage medium included in the exosuit 1900 by a variety of methods. Applications can be installed from a removable computer-readable storage medium or from a system in communication with the controller 1910 through the communications interface 1960. In some examples, the applications can be installed from a web site, a repository of compiled or un-compiled programs on the Internet, an online store (e.g., Google Play, iTunes App Store), or some other source. Further, functions of the applications can be contingent upon the controller 1910 being in continuous or periodic communication with a remote system (e.g., to receive updates, authenticate the application, to provide information about current environmental conditions).

The exosuit 1900 illustrated in FIG. 19 is intended as an illustrative example. Other configurations of flexible exosuits and of operating systems, kernels, applications, drivers, services, daemons, or other computer-readable programs are anticipated. For example, an operating system configured to operate an exosuit can include a real-time operating system component configured to generate low-level commands to operate elements of the exosuit and a non-real-time component to enable less time-sensitive functions, like a clock on a user interface, updating computer-readable programs stored in the exosuit, or other functions. A exosuit can include more than one controller; further, some of those controllers can be configured to execute real-time applications, operating systems, drivers, or other computer-readable programs (e.g., those controllers were configured to have very short interrupt servicing routines, very fast thread switching, or other properties and functions relating to latency-sensitive computations) while other controllers are configured to enable less time-sensitive functions of a flexible exosuit. Additional configurations and operating modes of an exosuit are anticipated. Further, control systems configured as described herein can additionally or alternatively be configured to enable the operation of devices and systems other than exosuit; for example, control systems as described herein can be configured to operate robots, rigid exosuits or exoskeletons, assistive devices, prosthetics, or other mechatronic devices.

Control of actuators of an exosuit can be implemented in a variety of ways according to a variety of control schemes. Generally, one or more hardware and/or software controllers can receive information about the state of the flexible exosuit, a wearer of the exosuit, and/or the environment of the exosuit from sensors disposed on or within the exosuit and/or a remote system in communication with the exosuit. The one or more hardware and/or software controllers can then generate a control output that can be executed by actuators of the exosuit to affect a commanded state of the exosuit and/or to enable some other application. One or more software controllers can be implemented as part of an operating system, kernel, driver, application, service, daemon, or other computer-readable program executed by a processor included in the exosuit.

In some embodiments, a powered assistive exosuit intended primarily for assistive functions can also be adapted to perform exosuit functions. In one embodiment, an assistive exosuit similar to the embodiments described in U.S. Publication No. 2018/0056104, that is used for assistive functions may be adapted to perform exosuit functions. Embodiments of such an assistive exosuit typically include FLAs approximating muscle groups such as hip flexors, gluteal/hip extensors, spinal extensors, or abdominal muscles. In the assistive modes of these exosuits, these FLAs provide assistance for activities such as moving between standing and seated positions, walking, and postural stability. Actuation of specific FLAs within such an exosuit system may also provide stretching assistance. Typically, activation of one or more FLAs approximating a muscle group can stretch the antagonist muscles. For example, activation of one or more FLAs approximating the abdominal muscles might stretch the spinal extensors, or activation of one or more FLAs approximating gluteal/hip extensor muscles can stretch the hip flexors. The exosuit may be adapted to detect when the wearer is ready to initiate a stretch and perform an automated stretching regimen; or the wearer may indicate to the suit to initiate a stretching regimen.

It can be appreciated that assistive exosuits may have multiple applications. Assistive exosuits may be prescribed for medical applications. These may include therapeutic applications, such as assistance with exercise or stretching regimens for rehabilitation, disease mitigation or other therapeutic purposes. Mobility-assistance devices such as wheelchairs, walkers, crutches and scooters are often prescribed for individuals with mobility impairments. Likewise, an assistive exosuit may be prescribed for mobility assistance for patients with mobility impairments. Compared with mobility assistance devices such as wheelchairs, walkers, crutches and scooters, an assistive exosuit may be less bulky, more visually appealing, and conform with activities of daily living such as riding in vehicles, attending community or social functions, using the toilet, and common household activities.

An assistive exosuit may additionally function as primary apparel, fashion items or accessories. The exosuit may be stylized for desired visual appearance. The stylized design may reinforce visual perception of the assistance that the exosuit is intended to provide. For example, an assistive exosuit intended to assist with torso and upper body activities may present a visual appearance of a muscular torso and upper body. Alternatively, the stylized design may be intended to mask or camouflage the functionality of the assistive exosuit through design of the base layer, electro/mechanical integration or other design factors.

Similarly to assistive exosuits intended for medically prescribed mobility assistance, assistive exosuits may be developed and utilized for non-medical mobility assistance, performance enhancement and support. For many, independent aging is associated with greater quality of life, however activities may become more limited with time due to normal aging processes. An assistive exosuit may enable aging individuals living independently to electively enhance their abilities and activities. For example, gait or walking assistance could enable individuals to maintain routines such as social walking or golf. Postural assistance may render social situations more comfortable, with less fatigue. Assistance with transitioning between seated and standing positions may reduce fatigue, increase confidence, and reduce the risk of falls. These types of assistance, while not explicitly medical in nature, may enable more fulfilling, independent living during aging processes.

Athletic applications for an assistive exosuit are also envisioned. In one example, an exosuit may be optimized to assist with a particular activity, such as cycling. In the cycling example, FLAs approximating gluteal or hip extensor muscles may be integrated into bicycle clothing, providing assistance with pedaling. The assistance could be varied based on terrain, fatigue level or strength of the wearer, or other factors. The assistance provided may enable increased performance, injury avoidance, or maintenance of performance in the case of injury or aging. It can be appreciated that assistive exosuits could be optimized to assist with the demands of other sports such as running, jumping, swimming, skiing, or other activities. An athletic assistive exosuit may also be optimized for training in a particular sport or activity. Assistive exosuits may guide the wearer in proper form or technique, such as a golf swing, running stride, skiing form, swimming stroke, or other components of sports or activities. Assistive exosuits may also provide resistance for strength or endurance training. The provided resistance may be according to a regimen, such as high intensity intervals.

Assistive exosuit systems as described above may also be used in gaming applications. Motions of the wearer, detected by the suit, may be incorporated as a game controller system. For example, the suit may sense wearer's motions that simulate running, jumping, throwing, dancing, fighting, or other motions appropriate to a particular game. The suit may provide haptic feedback to the wearer, including resistance or assistance with the motions performed or other haptic feedback to the wearer.

Assistive exosuits as described above may be used for military or first responder applications. Military and first responder personnel are often to be required to perform arduous work where safety or even life may be at stake. An assistive exosuit may provide additional strength or endurance as required for these occupations. An assistive exosuit may connect to one or more communication networks to provide communication services for the wearer, as well as remote monitoring of the suit or wearer.

Assistive exosuits as described above may be used for industrial or occupational safety applications. Exosuits may provide more strength or endurance for specific physical tasks such as lifting or carrying or repetitive tasks such as assembly line work. By providing physical assistance, assistive exosuits may also help avoid or prevent occupational injury due overexertion or repetitive stress.

Assistive exosuits as described above may also be configured as home accessories. Home accessory assistive exosuits may assist with household tasks such as cleaning or yard work, or may be used for recreational or exercise purposes. The communication capabilities of an assistive exosuit may connect to a home network for communication, entertainment or safety monitoring purposes.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art can appreciate that the conception, upon which this disclosure is based, may readily be utilized as a basis for the designing of other structures, systems, methods and media for carrying out the several purposes of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. An exosuit system, comprising:
an exosuit comprising a next-2-skin layer, a power layer, and a plurality of sensors, wherein the exosuit is operative to provide the plurality of assistive movements;
control circuitry coupled to the power layer and the plurality of sensors, the control circuitry operative to:
execute a body physiology estimator that obtains movement factors of a user of the exosuit;
execute a plurality of suit control modules based, at least in part, on the estimated movement factors and a selected one of the plurality of suit control module to control the plurality of assistive movements in accordance with parameters defined by the selected suit control module.

2. The exosuit system of claim 1, wherein the movement factors comprise bio-mechanical movement factors, wherein for any generic movement, a plurality of bio-mechanical movement factors define attributes of that generic movement.

3. The exosuit system of claim 1, wherein one of the plurality of suit control modules comprises an injury detection module that operates in conjunction with the control circuitry to:
determine whether the user has sustained an injury by analyzing the movement factors; and
notify the user the user that an injury has been detected.

4. The exosuit system of claim 1, wherein one of the plurality of suit control modules comprises an injury detection module that operates in conjunction with the control circuitry to:
establish a baseline of movement factors for a user of the exosuit;
monitor the plurality of sensors to obtain movement data while the user is using the exosuit;
analyze the movement data to obtain real-time movement factors;
in response to a first determination that a difference exists between the real-time movement factors and the baseline movement factors, wherein the first determination is indicative of an injury that cannot be compensated by exosuit assistance, notify the user that an injury has been detected; and
in response to a second determination that a difference exists between the real-time movement factors and the baseline movement factors, wherein the second determination is indicative of an injury that can be compensated by exosuit assistance, activate the power layer to compensate for the injury.

5. The exosuit system of claim 1, wherein one of the plurality of suit control modules comprises a training module that operates in conjunction with the control circuitry to:
receive a user selection of one of a plurality of training movements;
compare the movement factors obtained while the user performs the selected training movement; and
provide training feedback to the user based on the comparison, wherein training feedback comprises selective activation of the power layer to instruct the user of the correct training movement.

6. The exosuit system of claim 1, wherein one of the plurality of suit control modules comprises a productivity monitoring module that operates in conjunction with the control circuitry to assess productivity of workers using the exosuit.

7. The exosuit system of claim 1, wherein one of the plurality of suit control modules comprises an equipment operating module that operates in conjunction with the control circuitry to:
monitor at least the plurality of sensors to obtain equipment use data;
evaluate the equipment use data and the movement factors to determine whether the equipment is being used properly; and
if the equipment is deemed not being used properly:
activate the power layer to correct machine use; or
provide feedback to a user of the exosuit informing the user of incorrect equipment usage.

8. The exosuit system of claim 1, wherein one of the plurality of suit control modules comprises an equipment operating module that operates in conjunction with the control circuitry to:
monitor at least one of the sensors for a vibration event during use of equipment;
determine whether the vibration event exhibits a magnitude that exceeds a threshold that has been sustained for a minimum amount of time; and
provide feedback to user of the equipment based on the determination.

9. The exosuit system of claim 1, wherein one of the plurality of suit control modules comprises a policy and law enforcement module that operates in conjunction with the control circuitry to enforce a work place policy and/or laws for employees who use the exosuit.

10. A method for monitoring worker productivity, wherein the workers wear exosuits comprising a next-2-skin layer, a power layer, and a plurality of sensors, wherein the exosuit is operative to provide the plurality of assistive movements, the method comprising:
monitoring a plurality of workers each wearing the exosuit over a plurality of work shifts to establish a baseline productivity level for a typical worker using the exosuit for a typical work shift;
monitoring the plurality of workers to obtain work specific productivity data; and
in response to determining that a particular worker has work specific productivity data that is below the baseline productivity level, the method further comprises at least one of:
providing an alert to the particular worker if the exosuit is not functioning properly;
informing the particular work that an injury may have been sustained if movement factors obtained from the exosuit of the particular worker indicate occurrence of the injury; and
providing feedback to the particular work that productivity is too low.

11. The method of claim 10, wherein in response to determining that a particular worker has work specific productivity data that is below the baseline productivity level, the method further comprises notifying management that the particular worker has productivity that is too low.

12. The method of claim 10, wherein in response to determining that a particular worker has work specific productivity data that is below the baseline productivity level, the method further comprises informing the particular worker to take a training course when it is determined that the particular worker is not performing movements within optimal movement parameters.

13. The method of claim 10, wherein in response to determining that a particular worker has work specific productivity data that is below the baseline productivity level, the method further comprises adjusting at least one of the plurality of assistive movements.

14. The method of claim 10, wherein in response to determining that a particular worker has work specific productivity data that is below the baseline productivity level, the method further comprises instructing another worker to replace the particular worker.

15. An exosuit system, comprising:
an exosuit comprising a next-2-skin layer, a power layer, and a plurality of sensors, wherein the exosuit is operative to provide the plurality of assistive movements;
control circuitry coupled to the power layer and the plurality of sensors, the control circuitry operative to:
obtain movement factors of an exosuit user, wherein the movement factors are collected over a period of time to establish an exosuit assistance base line;
control the plurality of assistive movements according to the exosuit assistance base line;
determine that at least one of the movement factors has changed from a value used to establish the exosuit assistance baseline; and
alter operation of at least one of plurality of assistive movements to account for the movement factor determined to have changed.

16. The exosuit system of claim 15, wherein the control circuitry is operative to:
determine a fatigue level associated with the at least one of the movement factors determined to have changed from the exosuit assistance baseline; and
control operation of at least one of the plurality of assistive movement to a level that is proportional to the fatigue level.

17. The exosuit system of claim 15, wherein the control circuitry is operative to:
determine an injury has been sustained by the exosuit user based on a change in at least one of the movement factors; and
inform the exosuit user or a third party of the injury.

18. An exosuit system, comprising:
an exosuit comprising a next-2-skin layer, a power layer, and a plurality of sensors, wherein the exosuit is operative to provide the plurality of assistive movements;
control circuitry coupled to the power layer and the plurality of sensors, the control circuitry operative to:
monitor worker movement via the exosuit worn by the worker;
operate the exosuit to assist the worker in a work-related movement;
adjust operation of the exosuit to enable the worker to perform the work-related movement within a predefined range of parameters associated with the work-related movement; and
cease operating the exosuit when the work-related movement is determined to be complete.

19. The exosuit system of claim 18, wherein the work-related movement is a lifting movement of an object that can only be handled by the worker with the assistance of the exosuit.

20. The exosuit of claim 18, wherein the work-related movement is a repetitive movement.
